(19) United States
(12) Patent Application Publication
(10) Pub. No.: US 2025/0249019 A1
(43) Pub. Date: Aug. 7, 2025

(54) METHOD OF TREATMENT INCLUDING KRAS G12C INHIBITORS AND AURORA A INHIBITORS

(71) Applicant: ELI LILLY AND COMPANY, Indianapolis, IN (US)

(72) Inventors: Serge Louis BOULET, Fishers, IN (US); Xueqian GONG, Carmel, IN (US); Deqi GUO, Carmel, IN (US); David Michael HYMAN, Westport, CT (US); Sheng-Bin PENG, Carmel, IN (US); Chong SI, Zionsville, IN (US)

(21) Appl. No.: 18/854,022

(22) PCT Filed: Apr. 6, 2023
(86) PCT No.: PCT/US2023/065415

§ 371 Date: Oct. 3, 2024

Related U.S. Application Data
(60) Provisional application No. 63/329,147, filed on Apr. 8, 2022.

(51) Int. Cl. A61K 31/553; A61K 31/496; A61K 31/4985; A61P 35/00

(52) U.S. Cl. CPC A61K 31/553; A61K 31/496; A61K 31/4985; A61P 35/00

(57) ABSTRACT

The present disclosure provides method of treating a patient for cancer wherein one or more cells express KRas G12C mutant protein, comprising administering to a patient in need thereof, effective amounts of a compound of the formula:

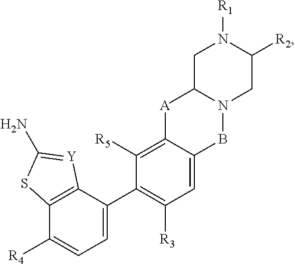

where R₁, R₂, R₃, R₄, R₅, R₆, R₇, R₈, R₉, R₁₀, R₁₁, R₁₂, A, B, and Y are as described herein, or pharmaceutically acceptable salts thereof, and an Aurora A inhibitor, or a pharmaceutically acceptable salt thereof.

The present disclosure relates to a method of treating a patient for cancer wherein one or more cells express KRas G12C mutant protein, comprising administering to a patient in need thereof, an effective amount of a KRAS G12C inhibitor, or pharmaceutically acceptable salts thereof with an Aurora A kinase inhibitor, or pharmaceutically acceptable salts thereof, to treat cancers such as lung cancer, colorectal cancer, pancreatic cancer, bladder cancer, cervical cancer, endometrial cancer, ovarian cancer, cholangiocarcinoma or esophageal cancer.

Oncogenic KRas mutations have been identified in approximately 30% of human cancers and have been demonstrated to activate multiple downstream signaling pathways. Despite the prevalence of KRas mutations, it has been a difficult therapeutic target. (Cox, A. D. Drugging the Undruggable RAS: Mission Possible?Nat. Rev. Drug Disc. 2014, 13, 828-851; Pylayeva-Gupta, y et al. RAS Oncogenes: Weaving a Tumorigenic Web. Nat. Rev. Cancer 2011, 11, 761-774).

WO2015/054572 and WO2016/164675 disclose certain quinazoline derivatives capable of binding to KRAS G12C. WO2016/044772 also discloses methods of using such quinazoline derivatives. WO2020/0081282 and WO2021/118877 disclose KRAS G12C inhibitors. WO2018/206539 and WO2020/178282 disclose certain heteroaryl compounds capable of binding to KRAS G12C oncoproteins.

The Aurora kinases are composed of 3 highly conserved serine/threonine isoforms, Aurora A, B, and C, which regulate mitotic and meiotic processes and function distinctly in cell cycle progression. Inhibition of Aurora A kinase activity significantly impairs mitotic progression through activation of the mitotic checkpoint, which results in defects in mitotic spindle formation and prometaphase arrest, with subsequent cell death through proapoptotic pathways. Amplification or overexpression of oncogenic Aurora A gene, AURKA, has been observed in a broad range of cancers.

Aurora A inhibitors are also known in the art. WO 2008/026768, EP 2062887, and WO2009/104802 disclose certain aminopyridine compounds having Aurora A selective inhibitory action. WO 2013/129443 discloses certain piperidine compounds having Aurora A selective inhibitory activity. WO2016/077161 and WO2020/112514 disclose certain aminopyridine compounds that inhibit Aurora A.

### (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:2-methylpropan-2-amine (1:1) salt

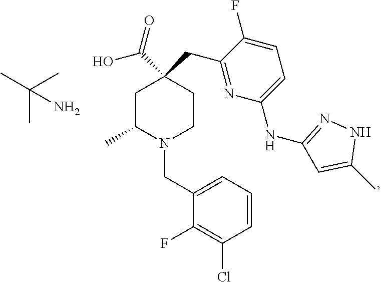

(“Aurora A Inh”) is a potent and selective inhibitor of Aurora A.

Tumor growth arrest or regression in a number of tumor types was observed following administration of (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:2-methylpropan-2-amine (1:1) salt to mice implanted with xenograft as well as PDX models as described in WO 2016/077161 and WO2020/112514.

In vitro studies of the combined inhibition of KRAS G12C and Aurora A using commercially available inhibitors demonstrate a synergistic antiproliferative effect and an antitumor effect. See Lito et al., Rapid non-uniform adaptation to conformation-specific KRAS G12C inhibition, Nature. 2020 January; 577(7790): 421-425.

There remains a need to provide small molecule combinations of KRAS G12C and Aurora A inhibitors. In particular, there is a need to provide more potent, orally deliverable KRAS G12C and Aurora A inhibitors that are useful for treating cancer. More particularly, there is a need to provide combinations of small molecule inhibitors that specifically inhibit KRas GTP and Aurora A activity. There is also a need to provide combinations of small molecule KRAS G12C and Aurora A inhibitors that exhibit synergistic antiproliferative effect and antitumor effect. Further, there is a desire to provide combinations of KRAS G12C and Aurora A inhibitors that overcome bypass of KRAS inhibition treatment. Also, there is a need to provide combinations of KRAS G12C and Aurora A inhibitors that exhibit increased efficacy with reduced or minimized untoward or undesired effects. The present disclosure addresses one or more of these needs by providing combinations and methods and uses for the combinations of KRAS G12C and Aurora A inhibitors.

The present disclosure provides a method of treating a patient for cancer wherein one or more cells express KRas G12C mutant protein, comprising administering to a patient in need thereof, an effective amount of a compound of Formula I:

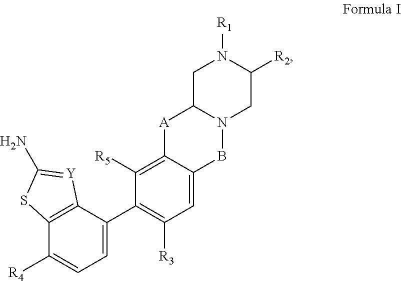

wherein:
A is —OCH₂—, —N(R₆)CH₂—, —OCH₂CH₂—, —N(R₆)CH₂CH₂—, —CH₂OCH₂—, or —CH₂N(R₆)CH₂—;
B is —CH₂— or —C(O)—;
Y is —C(CN)— or —N—;
R₁ is —CN, —C(O)C≡CR₈, or a group of the formula

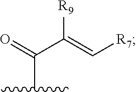

R₂ is H, methyl, or —CH₂CN;
R₃ and R₅ are each independently H, halogen, —C₀₋₃ alkyl-cyclopropyl, —C₁₋₆ alkyl optionally substituted 1-3 times with R₁₀, or —O—C₁₋₆ alkyl optionally substituted 1-3 times with R₁₀;
R₄ is H, halogen, or —C₁₋₆ alkyl optionally substituted 1-3 times with R₁₀;
R₆ is H or —C₁₋₆ alkyl optionally substituted 1-3 times with R₁₀;
R₇ is H, halogen, —NR₁₁R₁₂, —CH₂NR₁₁R₁₂, —C₁₋₆ alkyl optionally substituted 1-3 times with R₁₀ or R₁₃, —C₀₋₃ alkyl cyclopropyl, or —O—C₁₋₆ alkyl optionally substituted 1-3 times with R₁₀ or R₁₃;
R₈ is H, —C₁₋₄ alkyl optionally substituted 1-3 times with R₁₀, or —C₃₋₆ cycloalkyl optionally substituted 1-3 times with R₁₀;
R₉ is H, halogen, —CN, —C₀₋₃ alkyl-C₃₋₆ cycloalkyl, or —C₁₋₆ alkyl optionally substituted 1-3 times with R₁₀;
R₁₀ is independently at each occurrence halogen, oxygen, hydroxy, —C₁₋₄ alkyl, or —O—C₁₋₄ alkyl;
R₁₁ and R₁₂ are each independently H, —C₁₋₄ alkyl, or —C₁₋₄ heteroalkyl, wherein R₁₁ and R₁₂ may combine to form a heterocycloalkyl; and

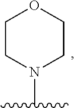 [SMILES: *N1CCOCC1]

R₁₃ is independently at each occurrence —N(CH₃)₂ or
or a pharmaceutically acceptable salt thereof; and
an effective amount of an Aurora A inhibitor, or a pharmaceutically acceptable salt thereof.

As used herein, the term halogen means fluoro (F), chloro (Cl), bromo (Br), or iodo (I). As used herein, the term alkyl means saturated linear or branched-chain monovalent hydrocarbon radicals of one to six carbon atoms, e.g., “—C₁₋₆ alkyl.” Examples of alkyls include, but are not limited to, methyl, ethyl, propyl, 1-propyl, isopropyl, butyl, pentyl, and hexyl. As used herein, the term heteroalkyl means saturated linear or branched-chain monovalent hydrocarbon radicals containing two to five carbon atoms and at least one heteroatom, e.g., “—C₁₋₄ heteroalkyl.” As used herein, the term cycloalkyl means saturated monovalent cyclic molecules with three to six carbon atoms, e.g., “—C₃₋₆ cycloalkyl.” Examples of cycloalkyl groups include, but are not limited to, cyclopropyl, cyclobutyl, cyclopentyl, and cyclohexyl. As used herein, the term cycloheteroalkyl means saturated monovalent cyclic molecules with two to five carbon atoms and at least one heteroatom, e.g., “—C₃₋₆ cycloheteroalkyl.” Examples of cycloheteroalkyl groups include, but are not limited to, pyrrolidine, piperidine, imidazolidine, pyrazolidine, and piperazine.

In cases where a zero is indicated, e.g., —C₀₋₃ alkyl-C₃₋₆ cycloalkyl, the alkyl component of the substituent group can be absent, thus, if R₉ of Formula I is a cyclopropyl group with no lead alkyl, the substituent would be described by the —C₀₋₃ alkyl-cyclopropyl substituent as described for R₉ (i.e., the substituent group would be —C₀-cyclopropyl).

Regarding R₁₁ and R₁₂, the two groups may combine with the nitrogen they are attached to when chemistry allows to form a heterocycloalkyl. Examples of said heterocycloalkyl groups include, but are not limited to, piperidine, piperazine, and morpholine.

In an embodiment the present disclosure provides a compound according to any one of Formula I or Formulae II-VI (see below), or a pharmaceutically acceptable salt thereof, for use in therapy in simultaneous, separate, or sequential combination with an Aurora A inhibitor, or a pharmaceutically acceptable salt thereof. The present disclosure also provides a compound according to any one of Formula I or Formulae II-VI, or a pharmaceutically acceptable salt thereof, for use in the treatment of cancer in simultaneous, separate, or sequential combination with an Aurora A inhibitor, or a pharmaceutically acceptable salt thereof. The present disclosure also provides for the use of a compound according to any one of Formula I or Formulae II-VI, or a pharmaceutically acceptable salt thereof, in the manufacture of a medicament for the treatment of cancer in simultaneous, separate, or sequential combination with an Aurora A kinase inhibitor, or a pharmaceutically acceptable salt thereof.

In an embodiment the present disclosure provides a method of treating a patient for cancer wherein one or more cells express KRas G12C mutant protein, comprising administering to a patient in need thereof, an effective amount of a compound of Formula I, or a pharmaceutically acceptable salt thereof, wherein the compound of Formula I is a compound of Formula Ia:

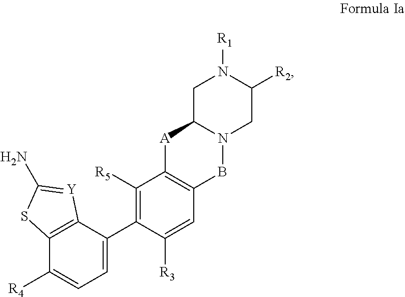

where R₁, R₂, R₃, R₄, R₅, A, B, and Y are as defined above, or a pharmaceutically acceptable salt thereof, and an effective amount of an Aurora A inhibitor, or a pharmaceutically acceptable salt thereof.

In an embodiment A is —OCH₂—, —N(R₆)CH₂—, —OCH₂CH₂—, —N(R₆)CH₂CH₂— in the compound of Formula I or Ia, or a pharmaceutically acceptable salt thereof. In a further embodiment A is —OCH₂— or —OCH₂CH₂— in the compound of Formula I or Ia, or a pharmaceutically acceptable salt thereof. In yet a further embodiment A is —OCH₂CH₂— in the compound of Formula I or Ia, or a pharmaceutically acceptable salt thereof.

In a further embodiment B is —C(O)— in the compound of Formula I or Ia, or a pharmaceutically acceptable salt thereof.

In a further embodiment Y is —C(CN)— in the compound of Formula I or Ia, or a pharmaceutically acceptable salt thereof.

In a further embodiment Y is —N— in the compound of Formula I or Ia, or a pharmaceutically acceptable salt thereof.

In a further embodiment R₁ is —CN, —C(O)C≡CR₈ in the compound of Formula I or Ia, or a pharmaceutically acceptable salt thereof. In yet a further embodiment R₁ is a group of the formula:

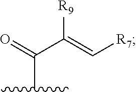

in the compound of Formula I or Ia, or a pharmaceutically acceptable salt thereof.

In a further embodiment R₂ is H or methyl in the compound of Formula I or Ia, or a pharmaceutically acceptable salt thereof. In yet a further embodiment R₂ is H in the compound of Formula I or Ia, or a pharmaceutically acceptable salt thereof.

In a further embodiment R₃ is H, halogen, methyl, methoxy, ethyl, isopropyl, or cyclopropyl in the compound of Formula I or Ia, or a pharmaceutically acceptable salt thereof. In yet a further embodiment R₃ is halogen, (preferably F or Cl) in the compound of Formula I or Ia, or a pharmaceutically acceptable salt thereof.

In a further embodiment R₄ is H or halogen in the compound of Formula I or Ia, or a pharmaceutically acceptable salt thereof. In yet a further embodiment R₄ is H, F, or Cl in the compound of Formula I or Ia, or a pharmaceutically acceptable salt thereof.

In a further embodiment R₅ is halogen (preferably Cl) in the compound of Formula I or Ia, or a pharmaceutically acceptable salt thereof.

In a further embodiment R₆ is H or CH₃ in the compound of Formula I or Ia, or a pharmaceutically acceptable salt thereof.

In a further embodiment R₉ is H, F, Cl, —CH₂F, —CF₃, or —CH₂OH in the compound of Formula I or Ia, or a pharmaceutically acceptable salt thereof. In yet a further embodiment R₉ is H in the compound of Formula I or Ia, or a pharmaceutically acceptable salt thereof.

In a further embodiment R₇ is H, —CHF₂, —CH₂F, —CH₂OH, —CH₂OCH₃, —CH₂N(CH₃)₂, or —CH₂-morpholine in the compound of Formula I or Ia, or a pharmaceutically acceptable salt thereof. In yet a further embodiment R₇ is H in the compound of Formula I or Ia, or a pharmaceutically acceptable salt thereof.

In yet a further embodiment R₉ is H and R₇ is H, —CHF₂, —CH₂F, —CH₂OH, —CH₂OCH₃, —CH₂N(CH₃)₂, or —CH₂-morpholine in the compound of Formula I or Ia, or a pharmaceutically acceptable salt thereof.

In yet a further embodiment R₉ is H, F, Cl, —CH₂F, —CF₃, or —CH₂OH in the compound of Formula I or Ia and R₇ is H in the compound of Formula I or Ia, or a pharmaceutically acceptable salt thereof.

In yet a further embodiment R₇ and R₉ are both H in the compound of Formula I or Ia, or a pharmaceutically acceptable salt thereof.

In yet a further embodiment R₁ is —CN, or —C(O)C≡CR₈ in the compound of Formula I or Ia and R₈ is H, methyl, —CH₂F, or —CH₂OH in the compound of Formula I or Ia, or a pharmaceutically acceptable salt thereof.

In yet a further embodiment R₁ is a group of the formula:

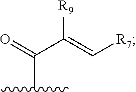

in the compound of Formula I or Ia,
and R₇ and R₉ are both H in the compound of Formula I or Ia, or a pharmaceutically acceptable salt thereof.

In yet a further embodiment R₁ is a group of the formula:

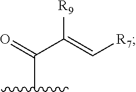

in the compound of Formula I or Ia,
and R₇ is tert-butyl in the compound of Formula I or Ia, and R₉ is —CN in the compound of Formula I or Ia, or a pharmaceutically acceptable salt thereof.

In yet a further embodiment A is —OCH₂—, —N(R₆)CH₂—, —OCH₂CH₂—, —N(R₆)CH₂CH₂— in the compound of Formula I or Ia, and B is —C(O)— in the compound of Formula I or Ia, or a pharmaceutically acceptable salt thereof.

In yet a further embodiment A is —OCH₂— or —OCH₂CH₂— in the compound of Formula I or Ia and B is —C(O)— in the compound of Formula I or Ia, or a pharmaceutically acceptable salt thereof.

In yet a further embodiment A is —OCH₂CH₂— in the compound of Formula I or Ia and B is —C(O)— in the compound of Formula I or Ia, or a pharmaceutically acceptable salt thereof.

In yet a further embodiment A is —OCH₂—, —N(R₆)CH₂—, —OCH₂CH₂—, or —N(R₆)CH₂CH₂— in the compound of Formula I or Ia, B is C(O) in the compound of Formula I or Ia, and R₂ is H or —CH₃ in the compound of Formula I or Ia, or a pharmaceutically acceptable salt thereof.

In yet a further embodiment A is —OCH₂— or —OCH₂CH₂—, B is —C(O)— in the compound of Formula I or Ia, and R₂ is H or methyl, in the compound of Formula I or Ia, or a pharmaceutically acceptable salt thereof.

In yet a further embodiment A is —OCH₂CH₂—, in the compound of Formula I or Ia, B is —C(O)— in the compound of Formula I or Ia, and R₂ is H or methyl, in the compound of Formula I or Ia, or a pharmaceutically acceptable salt thereof.

In yet a further embodiment A is —OCH₂—, —N(R₆)CH₂—, —OCH₂CH₂—, —N(R₆)CH₂CH₂—, in the compound of Formula I or Ia, B is —C(O)—, in the compound of Formula I or Ia, and R₂ is H, in the compound of Formula I or Ia, or a pharmaceutically acceptable salt thereof.

In yet a further embodiment A is —OCH₂— or —OCH₂CH₂—, in the compound of Formula I or Ia, B is —C(O)—, in the compound of Formula I or Ia, and R₂ is H, in the compound of Formula I or Ia, or a pharmaceutically acceptable salt thereof.

In yet a further embodiment A is —OCH₂CH₂—, in the compound of Formula I or Ia, B is —C(O)—, in the compound of Formula I or Ia, and R₂ is H, in the compound of Formula I or Ia, or a pharmaceutically acceptable salt thereof.

In yet a further embodiment A is —OCH₂CH₂—, in the compound of Formula I or Ia, and R₂ is H or methyl, in the compound of Formula I or Ia, or a pharmaceutically acceptable salt thereof.

In yet a further embodiment A is —OCH₂CH₂—, in the compound of Formula I or Ia, and R₂ is H, in the compound of Formula I or Ia, or a pharmaceutically acceptable salt thereof.

In yet a further embodiment B is —C(O)—, in the compound of Formula I or Ia, and R₂ is H or methyl, in the compound of Formula I or Ia, or a pharmaceutically acceptable salt thereof.

In yet a further embodiment B is —C(O)—, in the compound of Formula I or Ia, and R₂ is H, in the compound of Formula I or Ia, or a pharmaceutically acceptable salt thereof.

In yet a further embodiment R₃ and R₅ are each independently selected from H, halogen or methyl, in the compound of Formula I or Ia, or a pharmaceutically acceptable salt thereof.

In yet a further embodiment R₃ or R₅ are halogen, in the compound of Formula I or Ia, or a pharmaceutically acceptable salt thereof.

In yet a further embodiment R₃ and R₅ are halogen, in the compound of Formula I or Ia, or a pharmaceutically acceptable salt thereof.

In yet a further embodiment R₃ and R₅ are each independently selected from F or Cl, in the compound of Formula I or Ia, or a pharmaceutically acceptable salt thereof.

In yet a further embodiment Y is —C(CN)—, in the compound of Formula I or Ia, and R₄ is H or halogen (preferably F or Cl), in the compound of Formula I or Ia, or a pharmaceutically acceptable salt thereof.

In yet a further embodiment Y is —N—, in the compound of Formula I or Ia, and R₄ is H or halogen (preferably F or Cl), in the compound of Formula I or Ia, or a pharmaceutically acceptable salt thereof.

In yet a further embodiment Y is —C(CN)—, in the compound of Formula I or Ia, and R₃ and R₅ are each independently selected from methyl or halogen, in the compound of Formula I or Ia, or a pharmaceutically acceptable salt thereof.

In yet a further embodiment Y is —C(CN)—, in the compound of Formula I or Ia, and R₃ and R₅ are each halogen (preferably F or Cl), in the compound of Formula I or Ia, or a pharmaceutically acceptable salt thereof.

In yet a further embodiment Y is —N—, in the compound of Formula I or Ia, R₃ and R₅ are each independently selected from methyl or halogen, in the compound of Formula I or Ia, or a pharmaceutically acceptable salt thereof.

In yet a further embodiment Y is —N—, in the compound of Formula I or Ia, R₃ and R₅ are each halogen (preferably F or Cl), in the compound of Formula I or Ia, or a pharmaceutically acceptable salt thereof.

In yet a further embodiment A is —OCH₂—, —OCH₂CH₂—, —N(R₆)CH₂CH₂—, —CH₂OCH₂—, or —CH₂N(R₆)CH₂, in the compound of Formula I or Ia; B is —CH₂— or —C(O)—, in the compound of Formula I or Ia; Y is —C(CN)— or —N—, in the compound of Formula I or Ia; R₁ is —CN, —C(O)C≡CR₈, or a group of the formula:

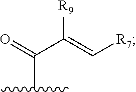

in the compound of Formula I or Ia;
R₂ is H or methyl, in the compound of Formula I or Ia; R₃ and R₅ are each H, F, Cl or methyl, in the compound of Formula I or Ia; R₄ is H or F; R₆ is H or methyl, in the compound of Formula I or Ia; R₇ is H, —CHF₂, —CH₂F, —CH₂OH, —CH₂OCH₃, —CH₂N(CH₃)₂, —CH₂-morpholine or tert-butyl, in the compound of Formula I or Ia; R₈ is methyl, —CH₂F or —CH₂OH, in the compound of Formula I or Ia; and R₉ is H, F, Cl, —CH₂F, —CF₃, —CH₂OH or CN, in the compound of Formula I or Ia; or a pharmaceutically acceptable salt thereof.

In yet a further embodiment A is —OCH₂— or —OCH₂CH₂—, in the compound of Formula I or Ia; B is —CH₂— or —C(O)—, in the compound of Formula I or Ia; Y is —C(CN)— or —N—, in the compound of Formula I or Ia; R₂, R₇, and R₈ are each H, in the compound of Formula I or Ia; R₄ is H or halogen, in the compound of Formula I or Ia; R₃ and R₅ are each halogen, in the compound of Formula I or Ia; or a pharmaceutically acceptable salt thereof.

The present disclosure further provides a method of treating a patient for cancer wherein one or more cells express KRas G12C mutant protein, comprising administering to a patient in need thereof, an effective amount of a compound of the Formula II:

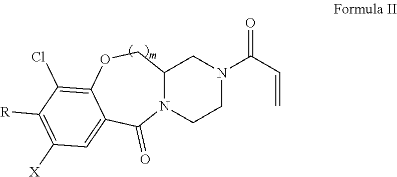

wherein R is

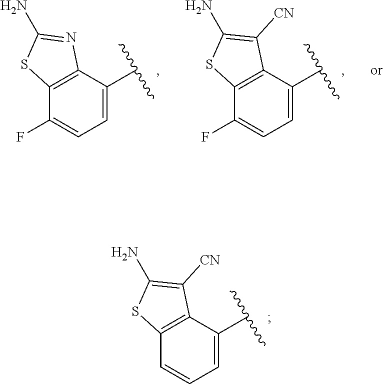 [SMILES: *c1ccc(F)c2sc(N)nc12 ; *c1ccc(F)c2sc(N)c(C#N)c12 ; *c1cccc2sc(N)c(C#N)c12]

X is CL of F;
and m is 1 or 2;
or a pharmaceutically acceptable salt thereof, and
an Aurora A inhibitor, or a pharmaceutically acceptable salt thereof.

The present disclosure also provides a method of treating a patient for cancer wherein one or more cells express KRas G12C mutant protein, comprising administering to a patient in need thereof, an effective amount of a compound of the Formula IIa:

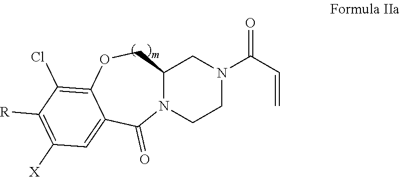

wherein R is

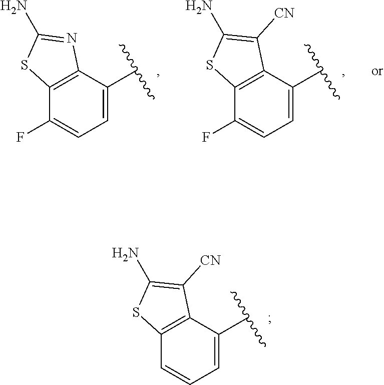 [SMILES: *c1ccc(F)c2sc(N)nc12 ; *c1ccc(F)c2sc(N)c(C#N)c12 ; *c1cccc2sc(N)c(C#N)c12]

X is Cl or F;
and m is 1 or 2;
or a pharmaceutically acceptable salt thereof, and
an Aurora A inhibitor, or a pharmaceutically acceptable salt thereof.

The present disclosure also provides a method of treating a patient for cancer wherein one or more cells express KRas G12C mutant protein, comprising administering to a patient in need thereof, an effective amount of a compound of Formula I, wherein the compound of Formula I is Formula Ib:

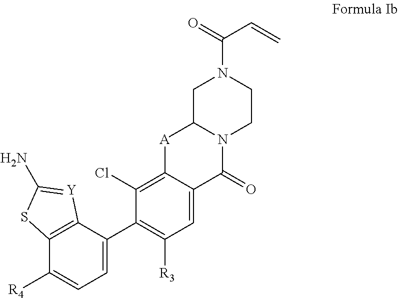

wherein:
A is —OCH₂— or —OCH₂CH₂—;
Y is —C(CN)— or —N—;
R₃ is Cl or F;
R₄ is H or F when Y is C(CN); and
R₄ is F when Y is N;
or a pharmaceutically acceptable salt thereof; and an effective amount of an Aurora A inhibitor, or a pharmaceutically acceptable salt thereof.

Another way to describe the compound of Formula IIa is with Formula Ib, wherein A is

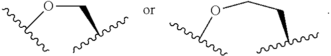

The present disclosure also provides a method of treating a patient for cancer wherein one or more cells express KRas G12C mutant protein, comprising administering to a patient in need thereof, an effective amount of a compound of Formula I or Ia selected from any one of Formulae III-VI below:

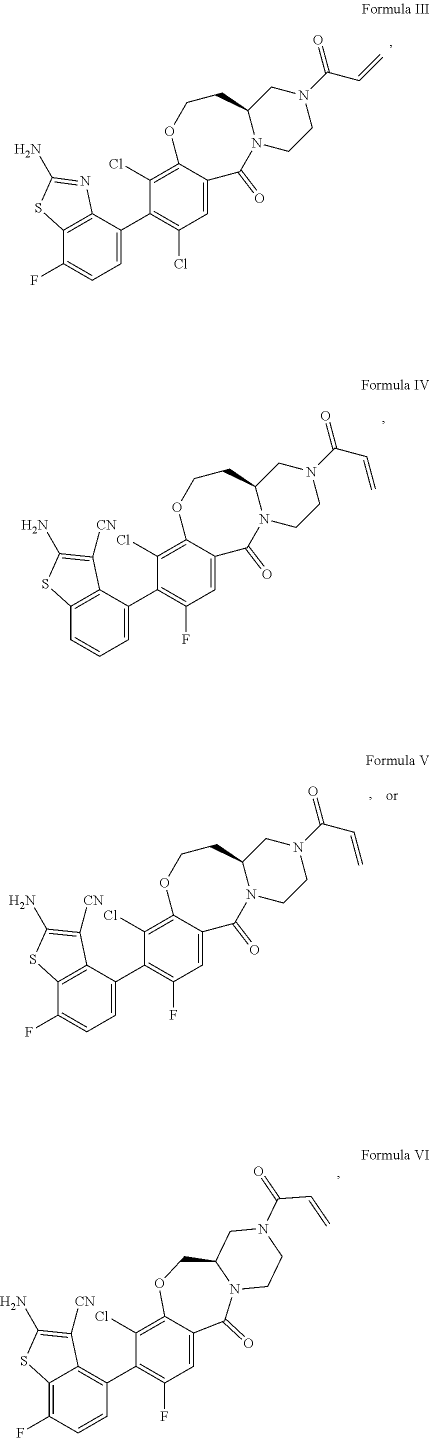 [SMILES: C=CC(=O)N1CCN2C(=O)c3cc(Cl)c(-c4ccc(F)c5sc(N)nc45)c(Cl)c3OCC[C@H]2C1 ; C=CC(=O)N1CCN2C(=O)c3cc(F)c(-c4cccc5sc(N)c(C#N)c45)c(Cl)c3OCC[C@H]2C1 ; C=CC(=O)N1CCN2C(=O)c3cc(F)c(-c4ccc(F)c5sc(N)c(C#N)c45)c(Cl)c3OCC[C@H]2C1 ; C=CC(=O)N1CCN2C(=O)c3cc(F)c(-c4ccc(F)c5sc(N)c(C#N)c45)c(Cl)c3OC[C@H]2C1]

or a pharmaceutically acceptable salt thereof, and
an Aurora A inhibitor, or a pharmaceutically acceptable salt thereof.

In another embodiment, the present disclosure provides a method of treating a patient for cancer wherein one or more cells express KRas G12C mutant protein, comprising administering to a patient in need thereof, an effective amount of a compound of Formula III which is:

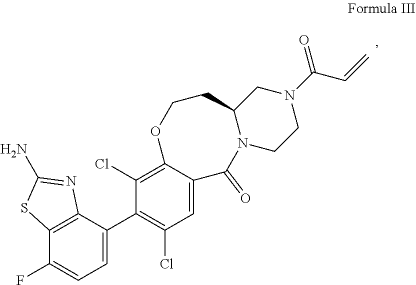 [SMILES: C=CC(=O)N1CCN2C(=O)c3cc(Cl)c(-c4ccc(F)c5sc(N)nc45)c(Cl)c3OCC[C@H]2C1]

or a pharmaceutically acceptable salt thereof, and
an Aurora A inhibitor, or a pharmaceutically acceptable salt thereof.

In another embodiment, the present disclosure provides a method of treating a patient for cancer wherein one or more cells express KRas G12C mutant protein, comprising administering to a patient in need thereof, an effective amount of a compound of Formula IV which is:

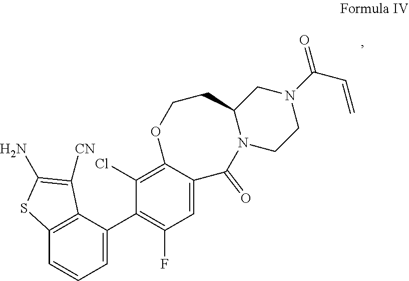 [SMILES: C=CC(=O)N1CCN2C(=O)c3cc(F)c(-c4cccc5sc(N)c(C#N)c45)c(Cl)c3OCC[C@H]2C1]

or a pharmaceutically acceptable salt thereof, and
an Aurora A inhibitor, or a pharmaceutically acceptable salt thereof.

In another embodiment, the present disclosure provides a method of treating a patient for cancer wherein one or more cells express KRas G12C mutant protein, comprising administering to a patient in need thereof, an effective amount of a compound of Formula V which is:

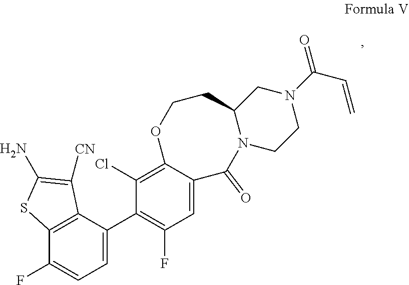 [SMILES: C=CC(=O)N1CCN2C(=O)c3cc(F)c(-c4ccc(F)c5sc(N)c(C#N)c45)c(Cl)c3OCC[C@H]2C1]

or a pharmaceutically acceptable salt thereof, and
an Aurora A inhibitor, or a pharmaceutically acceptable salt thereof.

In another embodiment, the present disclosure provides a method of treating a patient for cancer wherein one or more cells express KRas G12C mutant protein, comprising administering to a patient in need thereof, an effective amount of a compound of Formula VI which is:

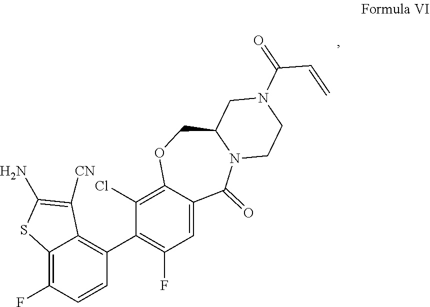 [SMILES: C=CC(=O)N1CCN2C(=O)c3cc(F)c(-c4ccc(F)c5sc(N)c(C#N)c45)c(Cl)c3OC[C@H]2C1]

or a pharmaceutically acceptable salt thereof, and
an Aurora A inhibitor, or a pharmaceutically acceptable salt thereof.

The present disclosure also provides a method of treating a patient for cancer wherein one or more cells express KRas G12C mutant protein, comprising administering to a patient in need thereof, an effective amount of a compound selected from the group consisting of:

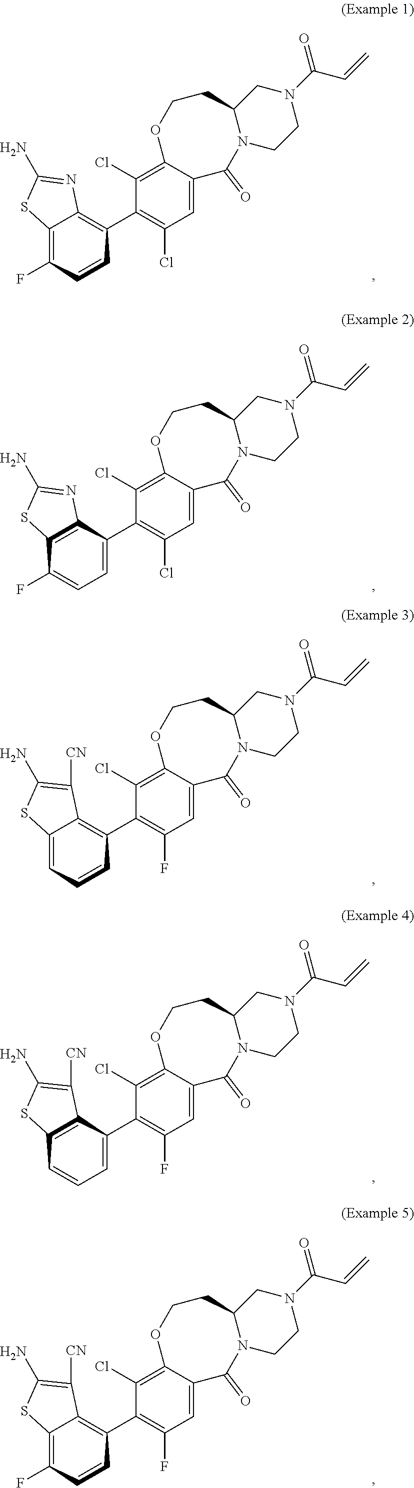

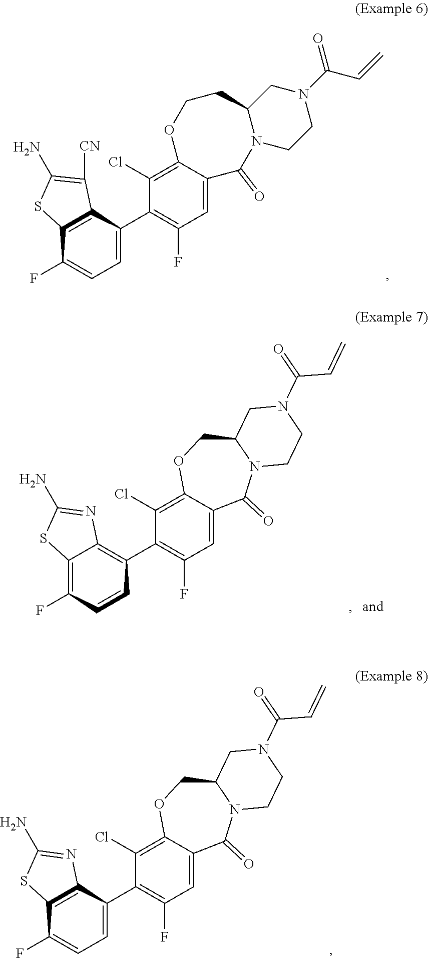

or a pharmaceutically acceptable salt thereof, and
an Aurora A inhibitor, or a pharmaceutically acceptable salt thereof.

The present disclosure also provides a method of treating a patient for cancer wherein one or more cells express KRas G12C mutant protein, comprising administering to a patient in need thereof, an effective amount of a compound selected from the group consisting of:

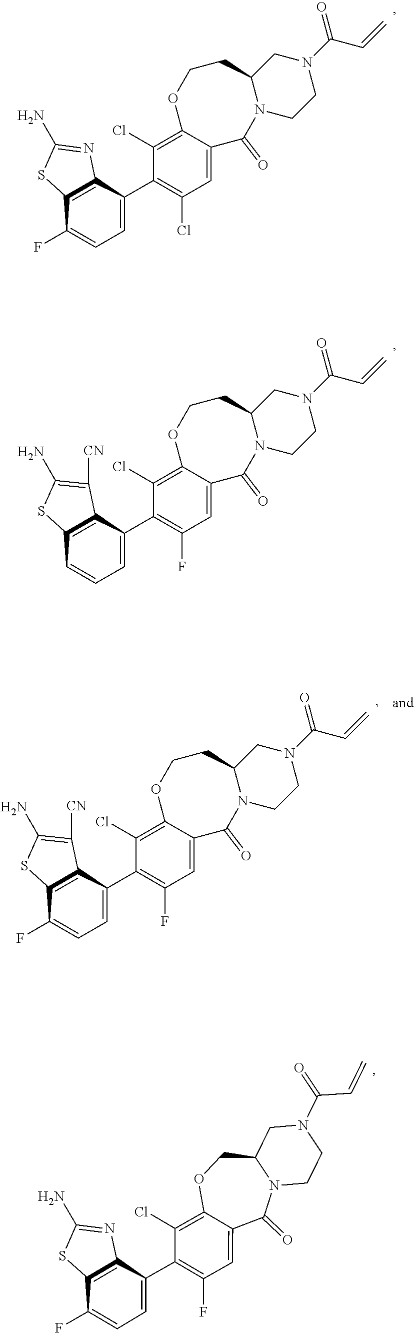

or a pharmaceutically acceptable salt thereof, and
an Aurora A inhibitor, or a pharmaceutically acceptable salt thereof.

The present disclosure also provides a method of treating a patient for cancer wherein one or more cells express KRas G12C mutant protein, comprising administering to a patient in need thereof, an effective amount of a compound selected from the group consisting of:

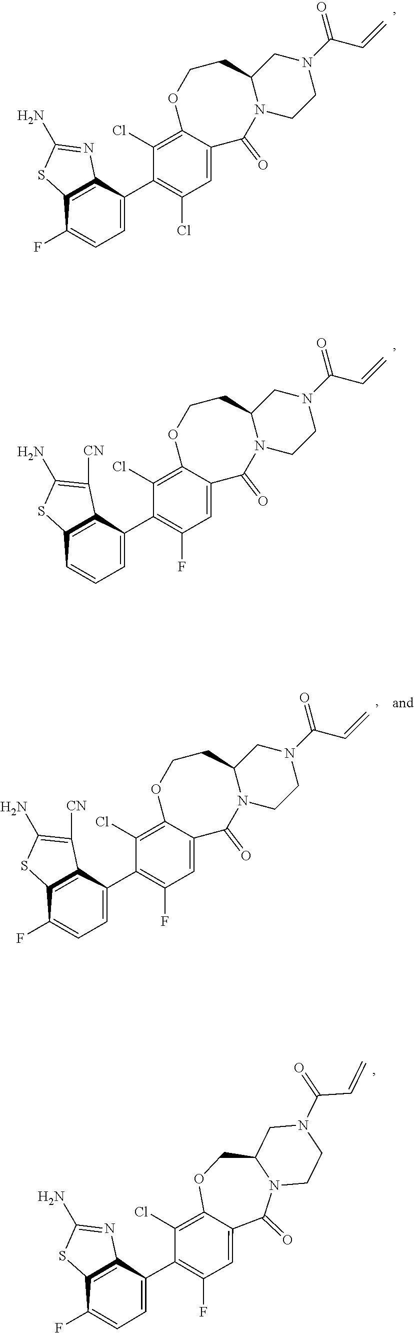

and
an Aurora A inhibitor, or a pharmaceutically acceptable salt thereof.

The present disclosure provides a method of treating a patient for cancer wherein one or more cells express KRas G12C mutant protein, comprising administering to a patient in need thereof, an effective amount of a compound selected from the group consisting of:

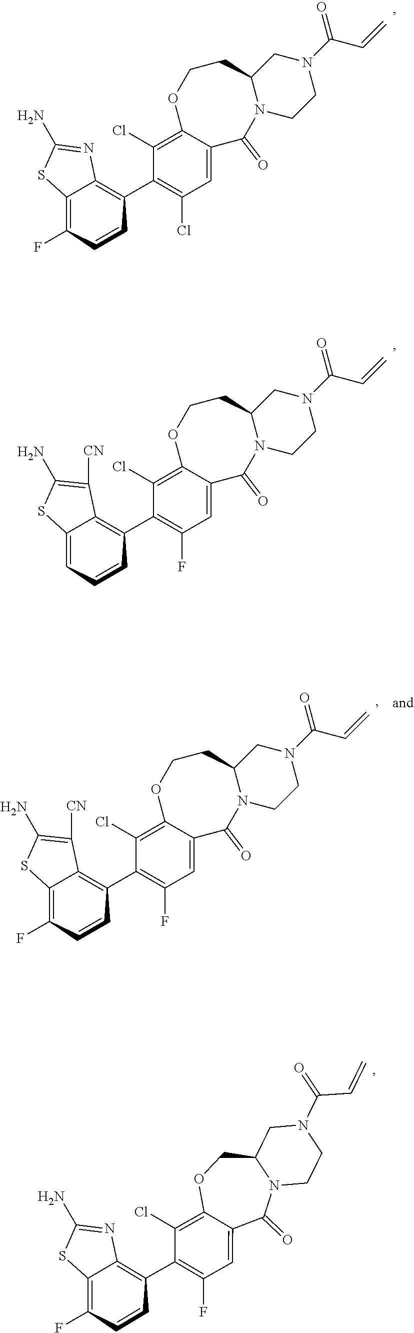

or a pharmaceutically acceptable salt thereof, and
an Aurora A inhibitor, or a pharmaceutically acceptable salt thereof.

The present disclosure provides a method of treating a patient for cancer wherein one or more cells express KRas G12C mutant protein, comprising administering to a patient in need thereof, an effective amount of a compound selected from the group consisting of:

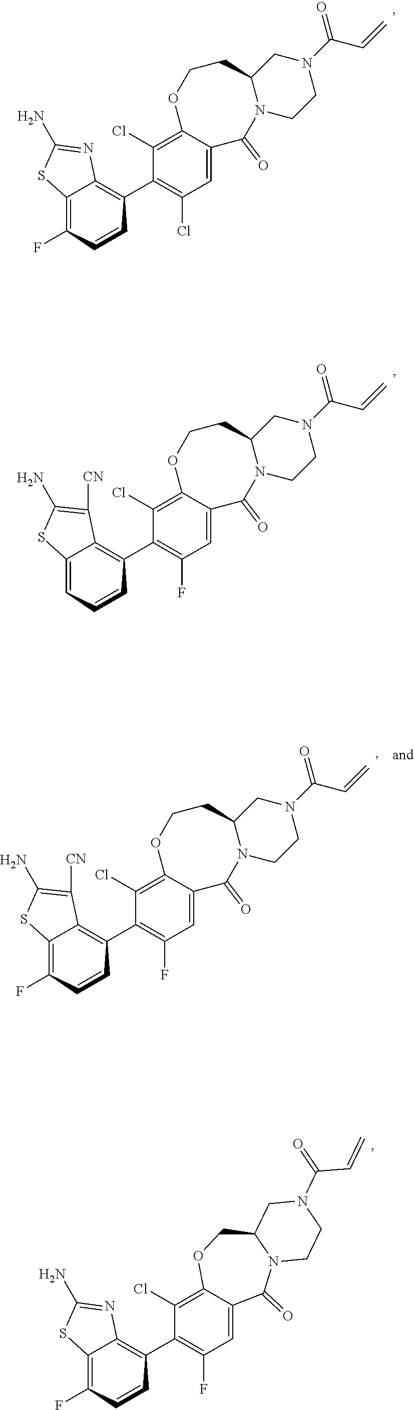

and
an Aurora A inhibitor, or a pharmaceutically acceptable salt thereof.

The present disclosure provides a method of treating a patient for cancer wherein one or more cells express KRas G12C mutant protein, comprising administering to a patient in need thereof, an effective amount of a compound selected from the group consisting of:

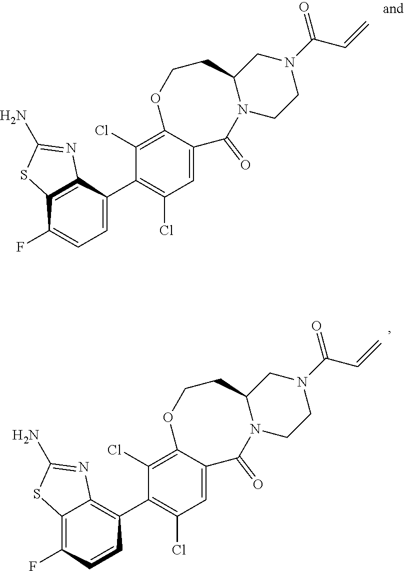

or a pharmaceutically acceptable salt thereof, and
an Aurora A inhibitor, or a pharmaceutically acceptable salt thereof.

The present disclosure provides a method of treating a patient for cancer wherein one or more cells express KRas G12C mutant protein, comprising administering to a patient in need thereof, an effective amount of a compound is

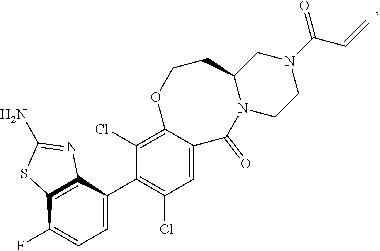

or a pharmaceutically acceptable salt thereof, and
   an Aurora A inhibitor, or a pharmaceutically acceptable salt thereof.

The present disclosure provides a method of treating a patient for cancer wherein one or more cells express KRas G12C mutant protein, comprising administering to a patient in need thereof, an effective amount of a compound is

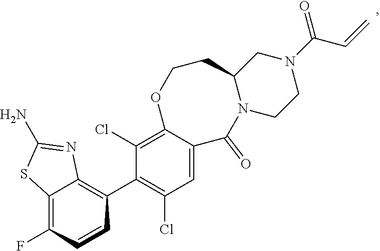

or a pharmaceutically acceptable salt thereof, and
   an Aurora A inhibitor, or a pharmaceutically acceptable salt thereof.

The present disclosure provides a method of treating a patient for cancer wherein one or more cells express KRas G12C mutant protein, comprising administering to a patient in need thereof, an effective amount of a compound selected from the group consisting of:

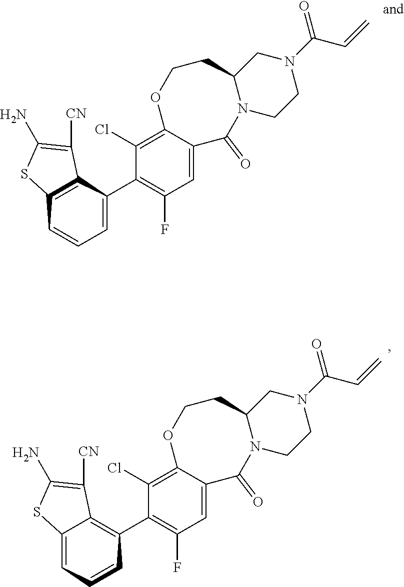

or a pharmaceutically acceptable salt thereof, and
an Aurora A inhibitor, or a pharmaceutically acceptable salt thereof.

The present disclosure provides a method of treating a patient for cancer wherein one or more cells express KRas G12C mutant protein, comprising administering to a patient in need thereof, an effective amount of a compound is

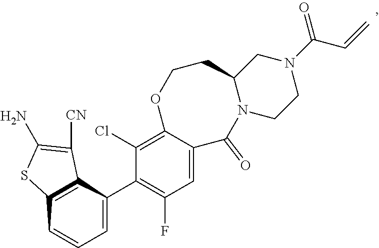

or a pharmaceutically acceptable salt thereof, and
   an Aurora A inhibitor, or a pharmaceutically acceptable salt thereof.

The present disclosure provides a method of treating a patient for cancer wherein one or more cells express KRas G12C mutant protein, comprising administering to a patient in need thereof, an effective amount of a compound is

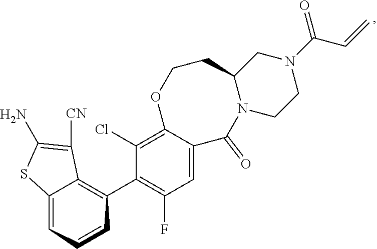

or a pharmaceutically acceptable salt thereof, and
an Aurora A inhibitor, or a pharmaceutically acceptable salt thereof.

The present disclosure provides a method of treating a patient for cancer wherein one or more cells express KRas G12C mutant protein, comprising administering to a patient in need thereof, an effective amount of a compound selected from the group consisting of:

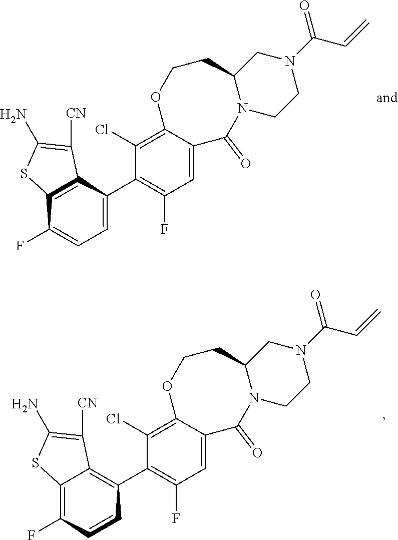

or a pharmaceutically acceptable salt thereof, and
an Aurora A inhibitor, or a pharmaceutically acceptable salt thereof.

The present disclosure provides a method of treating a patient for cancer wherein one or more cells express KRas G12C mutant protein, comprising administering to a patient in need thereof, an effective amount of a compound is

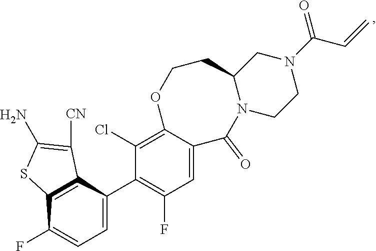

or a pharmaceutically acceptable salt thereof, and
   an Aurora A inhibitor, or a pharmaceutically acceptable salt thereof.

The present disclosure provides a method of treating a patient for cancer wherein one or more cells express KRas G12C mutant protein, comprising administering to a patient in need thereof, an effective amount of a compound is

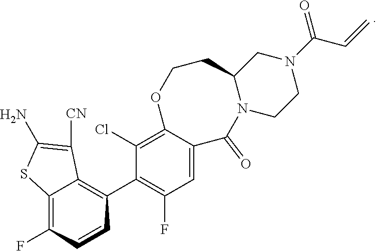

or a pharmaceutically acceptable salt thereof, and
an Aurora A inhibitor, or a pharmaceutically acceptable salt thereof.

The present disclosure provides a method of treating a patient for cancer wherein one or more cells express KRas G12C mutant protein, comprising administering to a patient in need thereof, an effective amount of a compound selected from the group consisting of:

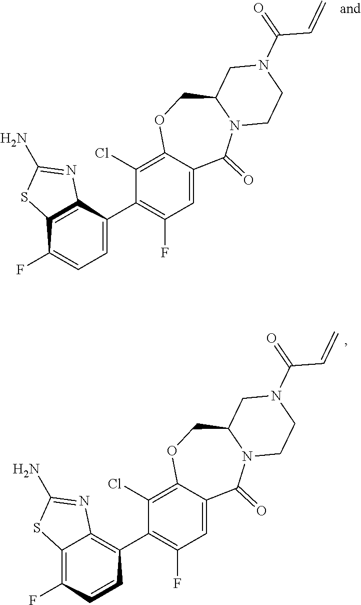

or a pharmaceutically acceptable salt thereof, and
an Aurora A inhibitor, or a pharmaceutically acceptable salt thereof.

The present disclosure provides a method of treating a patient for cancer wherein one or more cells express KRas G12C mutant protein, comprising administering to a patient in need thereof, an effective amount of a compound is

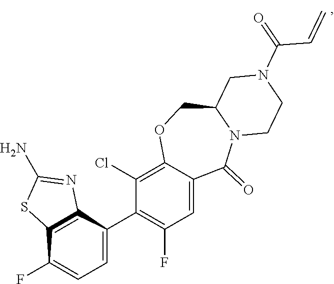

or a pharmaceutically acceptable salt thereof, and
   an Aurora A inhibitor, or a pharmaceutically acceptable salt thereof.

The present disclosure provides a method of treating a patient for cancer wherein one or more cells express KRas G12C mutant protein, comprising administering to a patient in need thereof, an effective amount of a compound is

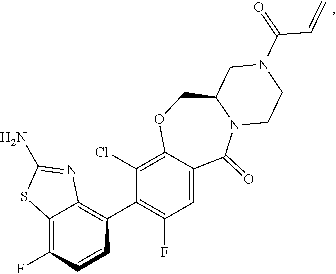

or a pharmaceutically acceptable salt thereof, and
an Aurora A inhibitor, or a pharmaceutically acceptable salt thereof.

In another embodiment, a method of treating a patient for cancer wherein one or more cells express KRas G12C mutant protein, comprising administering to a patient in need thereof, an effective amount of a compound according to any one of Formulae I-VI or Examples 1-8 also includes wherein the Aurora A inhibitor, or a pharmaceutically acceptable salt thereof, is an aminopyridine compound, or a pharmaceutically acceptable salt thereof. In another embodiment, the Aurora A inhibitor is an Aurora A selective inhibitor, or a pharmaceutically acceptable salt thereof. In another embodiment, the Aurora A inhibitor is alisertib as described in WO 2008/063525. In another embodiment, the Aurora A inhibitor is a pan Aurora inhibitor, or a pharmaceutically acceptable salt thereof. In another embodiment, the Aurora A inhibitor is tozasertib as described in WO 2004/000833. In another embodiment, the Aurora A inhibitor is danusertib as described in WO 2005/005427. In another embodiment, the Aurora A inhibitor is (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:

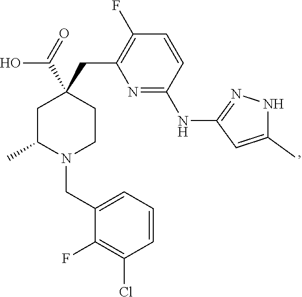

or a pharmaceutically acceptable salt thereof.

In another embodiment the present disclosure provides a method of treating a patient for cancer wherein one or more cells express KRas G12C mutant protein, comprising administering to a patient in need thereof, an effective amount of a compound according to any one of Formulae I-VI or Examples 1-8, or a pharmaceutically acceptable salt thereof, with (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid, or a pharmaceutically acceptable salt thereof.

In another embodiment the present disclosure provides a method of treating a patient for cancer wherein one or more cells express KRas G12C mutant protein, comprising administering to a patient in need thereof, an effective amount of a compound according to any one of Formulae I-VI or Examples 1-8, or a pharmaceutically acceptable salt thereof, with (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:amine (1:1) salt:

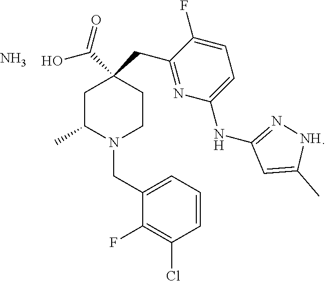

In another embodiment the present disclosure provides a method of treating a patient for cancer wherein one or more cells express KRas G12C mutant protein, comprising administering to a patient in need thereof, an effective amount of a compound according to any one of Formulae I-VI or Examples 1-8, or a pharmaceutically acceptable salt thereof, with (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:2-methylpropan-2-amine (1:1) salt:

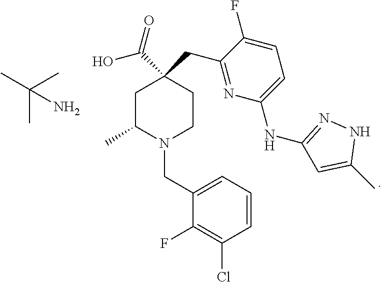

The present disclosure also provides a method of treating a patient for cancer wherein one or more cells express KRas G12C mutant protein, comprising administering to a patient in need thereof, an effective amount of a compound according to any one of Formulae I-VI or Examples 1-8, or a pharmaceutically acceptable salt thereof, with an Aurora A inhibitor, or a pharmaceutically acceptable salt thereof.

In various embodiments, the cancer is lung cancer, colorectal cancer, pancreatic cancer, bladder cancer, cervical cancer, endometrial cancer, ovarian cancer, cholangiocarcinoma, or esophageal cancer. In preferred embodiments, the cancer is non-small cell lung cancer, pancreatic cancer, or colorectal cancer. In still more preferred embodiments, the cancer is non-small cell lung cancer.

The present disclosure also provides a method of treating a patient with a cancer comprising administering to a patient in need thereof an effective amount of a compound according to any one of Formulae I-VI or Examples 1-8, or a pharmaceutically acceptable salt thereof, with an Aurora A kinase inhibitor, or a pharmaceutically acceptable salt thereof, in which the cancer has one or more cells that express a mutant KRas G12C protein with or without an Aurora A dysregulation or overexpression. The present disclosure also provides a method of treating cancer, comprising administering to a patient in need thereof, an effective amount of a compound according to any one of Formulae I-VI or Examples 1-8, or a pharmaceutically acceptable salt thereof, and an Aurora A kinase inhibitor compound, or a pharmaceutically acceptable salt thereof, wherein the cancer is non-small cell lung cancer, and wherein one or more cells with or without an Aurora A dysregulation or overexpression express KRas G12C mutant protein. The present disclosure also provides a method of treating cancer, comprising administering to a patient in need thereof, an effective amount of a compound according to any one of Formulae I-VI or Examples 1-8, or a pharmaceutically acceptable salt thereof, and an Aurora A kinase inhibitor compound, or a pharmaceutically acceptable salt thereof, wherein the cancer is colorectal cancer, and wherein one or more cells with or without an Aurora A dysregulation or overexpression express KRas G12C mutant protein. The present disclosure also provides a method of treating cancer, comprising administering to a patient in need thereof, an effective amount of a compound according to any one of Formulae I-VI or Examples 1-8, or a pharmaceutically acceptable salt thereof, and an Aurora A kinase inhibitor compound, or a pharmaceutically acceptable salt thereof, wherein the cancer is pancreatic cancer, and wherein one or more cells with or without an Aurora A dysregulation or overexpression express KRas G12C mutant protein.

The present disclosure also provides a method of treating cancer in a patient in need thereof, wherein the patient has a cancer that was determined to express the KRas G12C mutant protein and an Aurora A dysregulation or overexpression.

In another embodiment, the cancer is non-small cell lung carcinoma, in which the cancer has one or more cells that express a KRas G12C mutant protein and/or an Aurora A dysregulation or overexpression. In another embodiment, the cancer is colorectal carcinoma in which the cancer has one or more cells that express a KRas G12C mutant protein and/or an Aurora A dysregulation or overexpression. In yet another embodiment, the cancer is mutant pancreatic cancer in which the cancer has one or more cells that express a KRas G12C mutant protein and/or an Aurora A dysregulation or overexpression. In another embodiment, the present disclosure comprises a method of treating KRas G12C mutant bearing cancers of other origins and/or an Aurora A dysregulation or overexpression.

In still yet another embodiment, the present disclosure comprises a method of treating cancer, comprising administering to a patient in need thereof, an effective amount of a compound according to any one of Formulae I-VI or Examples 1-8, or a pharmaceutically acceptable salt thereof, with an Aurora A kinase inhibitor, or a pharmaceutically acceptable salt thereof, in which the cancer has one or more cells that express a mutant KRas G12C protein or an Aurora A dysregulation or overexpression. In some embodiments, the patient has a cancer that was determined to have one or more cells expressing the KRas G12C mutant protein prior to administration of the compound, or a pharmaceutically acceptable salt thereof, or the Aurora A inhibitor, or a pharmaceutically acceptable salt thereof. In some embodiments, the patient has a cancer that has a KRAS G12C mutation.

In still yet another embodiment, the present disclosure comprises a method of treating cancer, comprising administering to a patient in need thereof, an effective amount of a compound according to any one of Formulae I-VI or Examples 1-8, or a pharmaceutically acceptable salt thereof, with an Aurora A kinase inhibitor, or a pharmaceutically acceptable salt thereof, wherein the compound according to any one of Formulae I-VI or Examples 1-8 and the Aurora A inhibitor are administered in simultaneous or sequential combination to the patient in need thereof. In some embodiments, the compound according to any one of Formulae I-VI or Examples 1-8 and the Aurora A inhibitor are administered in simultaneous combination to the patient in need thereof. In some embodiments, the compound according to any one of Formulae I-VI or Examples 1-8 and the Aurora A inhibitor are administered in sequential combination to the patient in need thereof. In some embodiments, the compound according to any one of Formulae I-VI or Examples 1-8 is administered to the patient in need thereof before the Aurora A inhibitor is administered to the patient in need thereof. In some embodiments, the Aurora A inhibitor is administered to the patient in need thereof before the compound according to any one of Formulae I-VI or Examples 1-8 is administered to the patient in need thereof.

Preferably, the cancer is lung cancer, colorectal cancer, pancreatic cancer, bladder cancer, cervical cancer, endometrial cancer, ovarian cancer, cholangiocarcinoma, or esophageal cancer. In preferred embodiments, the cancer is non-small cell lung cancer, pancreatic cancer, or colorectal cancer. In still more preferred embodiments, the cancer is non-small cell lung cancer. In other embodiments, the cancer has one or more cancer cells that express the mutant KRas G12C protein and/or an Aurora A dysregulation or overexpression. Preferably, the cancer is selected from KRas G12C mutant non-small cell lung cancer, KRas G12C mutant colorectal cancer, and KRas G12C mutant pancreatic cancer.

The term “pharmaceutically acceptable salt” as used herein refers to a salt of a compound considered to be acceptable for clinical and/or veterinary use. Examples of pharmaceutically acceptable salts and common methodology for preparing them can be found in “Handbook of Pharmaceutical Salts: Properties, Selection and Use” P. Stahl, et al., 2nd Revised Edition, Wiley-VCH, 2011 and S. M. Berge, et al., “Pharmaceutical Salts”, Journal of Pharmaceutical Sciences, 1977, 66(1), 1-19.

The pharmaceutical compositions for the present disclosure may be prepared using pharmaceutically acceptable additives. The term “pharmaceutically acceptable additive(s)” as used herein for the pharmaceutical compositions, refers to one or more carriers, diluents, and excipients that are compatible with the other additives of the composition or formulation and not deleterious to the patient. Examples of pharmaceutical compositions and processes for their preparation can be found in “Remington: The Science and Practice of Pharmacy”, Loyd, V., et al. Eds., 22^{nd} Ed., Mack Publishing Co., 2012. Non-limiting examples of pharmaceutically acceptable carriers, diluents, and excipients include the following: saline, water, starch, sugars, mannitol, and silica derivatives; binding agents such as carboxymethyl cellulose, alginates, gelatin, and polyvinyl-pyrrolidone; kaolin and bentonite; and polyethyl glycols.

As used herein, the term “effective amount” refers to an amount that is effective in treating a disorder or disease, such as a cancerous lesion or progression of abnormal cell growth and/or cell division. The attending physician, as one skilled in the art, can readily determine an effective amount by the use of conventional techniques and by observing results obtained under analogous circumstances.

As used herein, the term “dose” refers to the total amount of a drug that is administered at one time. An example of a drug includes a compound of Formula I, or a pharmaceutically acceptable salt thereof. Another example of a drug includes an Aurora A kinase inhibitor compound, or a pharmaceutically acceptable salt thereof.

As used herein, the terms “dosage” or “dose frequency” refer to a dose administered at a specific frequency.

Dose per day of treatment for the compound of Formula I normally fall within a range of between about 1 mg per day or twice daily and 1000 mg per day or twice daily, more preferably 100 mg per day or twice daily and 900 mg per day or twice daily.

Dose per day of treatment for the Aurora A inhibitor, an aminopyridine compound, or a pharmaceutically acceptable salt thereof, or (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:2-methylpropan-2-amine (1:1) salt, and (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:amine (1:1) salt, or a pharmaceutically acceptable salt thereof, normally fall within the range of about 0.1 to about 100 mg. In some instances, dose levels below the lower limit of this range may be more than adequate, while in other cases still larger doses may be employed for the Aurora A inhibitor, an aminopyridine compound, or a pharmaceutically acceptable salt thereof, (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:2-methylpropan-2-amine (1:1) salt, or (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:amine (1:1) salt, or a pharmaceutically acceptable salt thereof. Preferred doses fall within the range of 1 to 80 mg; more preferably between 1 and 50 mg; still more preferably between 1 and 30 mg; still yet more preferably between 1 to 25 mg, for the Aurora A inhibitor, an aminopyridine compound, or a pharmaceutically acceptable salt thereof, (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:2-methylpropan-2-amine (1:1) salt, or (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:amine (1:1) salt, or a pharmaceutically acceptable salt thereof. The doses can be administered once, twice, three times or more daily. In one embodiment, the Aurora A inhibitor, an aminopyridine compound, or a pharmaceutically acceptable salt thereof, (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:2-methylpropan-2-amine (1:1) salt, or (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:amine (1:1) salt, or a pharmaceutically acceptable salt thereof, can be administered at a dosage of 15 mg or 25 mg per dose administered orally twice a day (BID). Factors considered in the determination of an effective amount or dose of a compound include: whether the compound or its salt will be administered; the co-administration of other agents, if used; the species of patient to be treated; the patient's size, age, gender, and general health; the degree of involvement or stage and/or the severity of the disorder; the response of the individual patient; the mode of administration; the bioavailability characteristics of the preparation administered; the dose regimen selected; and the use of other concomitant medication.

A treating physician, veterinarian, or other medical person will be able to determine an effective amount of the compound for treatment of a patient in need. Preferred pharmaceutical compositions can be formulated as a tablet or capsule for oral administration, a solution for oral administration, or an injectable solution. The tablet, capsule, or solution can include a compound of the present disclosure in an amount effective for treating a patient in need of treatment for cancer wherein one or more cells express KRas G12C mutant protein.

As used herein, the terms “treating”, “to treat”, or “treatment”, includes slowing, reducing, or reversing the progression or severity of an existing symptom, disorder, condition, which can include specifically slowing the growth of a cancerous lesion or progression of abnormal cell growth and/or cell division.

As used herein, the term “patient” refers to a mammal in need of treatment. Preferably, the patient is a human that is in need of treatment for cancer wherein one or more cells express KRas G12C mutant protein, for example, KRas G12C mutant bearing cancers.

Individual isomers, enantiomers, diastereomers, and atropisomers may be separated or resolved at any convenient point in the synthesis of compounds listed below, by methods such as selective crystallization techniques or chiral chromatography (See for example, J. Jacques, et al., “Enantiomers, Racemates, and Resolutions”, John Wiley and Sons, Inc., 1981, and E. L. Eliel and S. H. Wilen, “Stereochemistry of Organic Compounds”, Wiley-Interscience, 1994). The present disclosure includes certain compounds, which are atropisomers and which can exist in different conformations or as different rotomers. Atropisomers are compounds, which exist in different conformations arising from restricted rotation about a single bond. Atropisomers can be isolated as separate chemical species if the energy barrier to rotation about the single is sufficiently high enough and the rate of interconversion is slow enough to allow the individual rotomers to be separated from each other. The present disclosures contemplates all of the isomers, enantiomers, diastereomers, and atropisomers disclosed herein or that could be made using the compounds disclosed herein.

Any compound according to any one of Formulae I-VI or Examples 1-8 is readily converted to and may be isolated as a pharmaceutically acceptable salt. Salt formation can occur upon the addition of a pharmaceutically acceptable acid to form the acid addition salt. Salts can also form simultaneously upon deprotection of a nitrogen or oxygen, i.e., removing the protecting group. Examples, reactions and conditions for salt formation can be found in Gould, P. L., “Salt selection for basic drugs,” International Journal of Pharmaceutics, 33: 201-217 (1986); Bastin, R. J., et al. “Salt Selection and Optimization Procedures for Pharmaceutical New Chemical Entities,” Organic Process Research and Development, 4: 427-435 (2000); and Berge, S. M., et al., “Pharmaceutical Salts,” Journal of Pharmaceutical Sciences, 66: 1-19, (1977).

The compounds of the present disclosure, or salts thereof, may be prepared by a variety of procedures, some of which are illustrated in the Preparations and Examples of WO2021/118877, WO2016/077161, and WO2020/112514. The specific synthetic steps for each of the routes described may be combined in different ways, or in conjunction with steps from different routes, to prepare compounds or salts of the present disclosure. The products of each step in the Preparations below can be recovered by conventional methods, including extraction, evaporation, precipitation, chromatography, filtration, trituration, and crystallization.

Suitable reaction conditions for the steps of these Preparations and Example are well known in the art and appropriate substitutions of solvents and co-reagents are within the skill of the art. Likewise, it will be appreciated by those skilled in the art that synthetic intermediates may be isolated and/or purified by various well-known techniques as needed or desired, and that frequently, it will be possible to use various intermediates directly in subsequent synthetic steps with little or no purification. As an illustration, compounds of the preparations and examples can be isolated, for example, by silica gel purification, isolated directly by filtration, or crystallization. Furthermore, the skilled artisan will appreciate that in some circumstances, the order in which moieties are introduced is not critical. The particular order of steps required to produce the compounds of the present disclosure is dependent upon the particular compound being synthesized, the starting compound, and the relative liability of the substituted moieties, as is well appreciated by the skilled chemist. All substituents, unless otherwise indicated, are as previously defined, and all reagents are well known and appreciated in the art.

The specific synthetic steps for each of the routes described may be combined in different ways, or in conjunction with steps from different routes, to prepare compounds or salts of the present disclosure. The products of each step in the Preparations below can be recovered by conventional methods, including extraction, evaporation, precipitation, chromatography, filtration, trituration, and crystallization.

The compounds of the present disclosure can be prepared in a number of ways well known to those skilled in the art of organic synthesis. By way of example, compounds of the present disclosure can be synthesized using the methods described below, together with synthetic methods known in the art of synthetic organic chemistry, or variations thereon as appreciated by those skilled in the art. Preferred methods include but are not limited to those methods described below.

Compounds of the present disclosure can be synthesized in part by following the steps outlined in the following Schemes 1-1a which comprise different sequences of assembling intermediates or compounds. Starting materials are either commercially available or made by known procedures in the reported literature or as illustrated below.

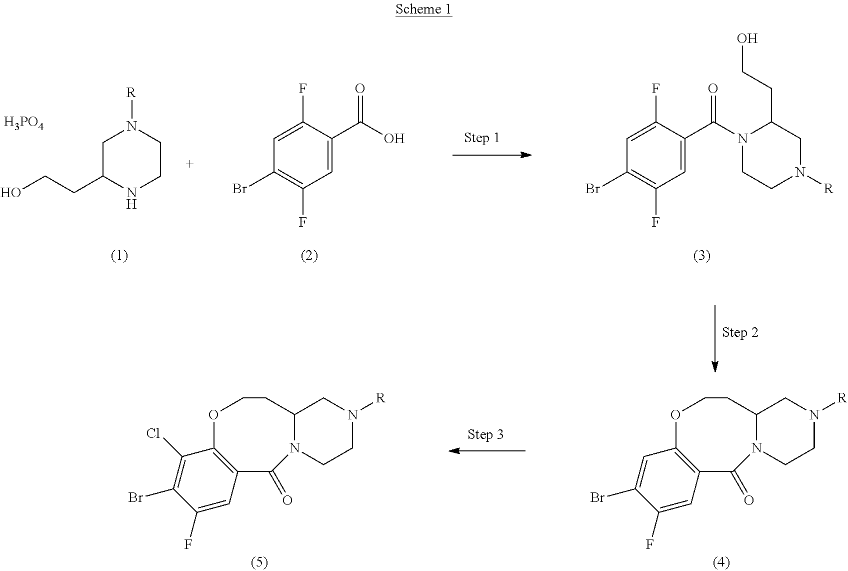

In Step 1 of Scheme 1, the protected piperazine-2-ethanol, compound (1), is coupled with 4-bromo-2,5-difluoro-benzoic acid, compound (2), in an amide bond formation using a coupling reagent such as CDMT with an organic base such as NMM in a solvent system such as acetonitrile and water and an inorganic base such as K₂CO₃. R is a protecting group developed for the amino group, such as carbamates and amides. Such protecting groups are well known and appreciated in the art, such as carbamate protecting groups including allyloxycarbonyl, fluorenylmethoxycarbonyl, or benzyloxycarbonyl. A common and preferred protecting group can be Boc. One skilled in the art will recognize that there are a number of methods and reagents for amide formation resulting from the reaction of carboxylic acids and amines. For example, the reaction of the amine compound with an appropriate carboxylic acid in the presence of a coupling reagent with or without an organic base such as DIPEA or TEA can provide compound (3). Other coupling reagents include carbodiimides, such as DCC, DIC, EDCI or a carbonyldiimidazole such as CDI. Amide coupling additives, such as HOBt and HOAt can also be used to enhance the reaction. Additionally, uronium or phosphonium salts of non-nucleophilic anions, such as HBTU, HATU, PyBOP, and PyBrOP could be used in place of the more traditional coupling reagents. An additive such as DMAP may be used to enhance the reaction. Alternatively, the acid chloride of compound (2) can be used in the presence of a base, such as TEA or pyridine to give compound (3).

In Step 2, the intramolecular cyclization of compound (3) is completed using an appropriate base such as potassium tert-butoxide, sodium tert-amylate, sodium tert-butoxide, sodium tert-pentoxide, DIPEA, TEA, DBU, sodium hydride in a solvent such as DMF to give compound 4. Other possible solvents could be NMP, DMAc, DMSO, and THF. This intramolecular cyclization of compound (3) to compound (4) may be conducted by slowly adding a solution of compound (3) to an excess of base so as to minimize intermolecular reaction derived impurities.

In Step 3, compound (4) can be chlorinated with under acidic conditions using an acid such as TFA, with a chlorinating agent such as trichloroisocyanuric acid or NCS in a solvent such as acetonitrile or DMF to give compound (5).

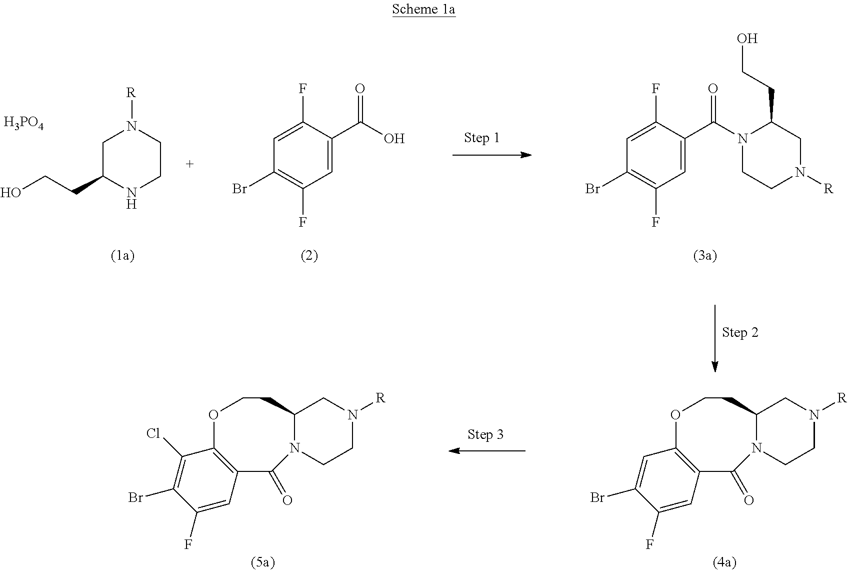

Scheme 1a illustrates a chiral synthesis of compound (5a). Compound (1a) can be prepared as described by Medicinal Chemistry route to 1, Development of an Alternative Route to the Bicyclic Piperazine, Retrosynthetic analysis of bicyclic piperazine core 2, and/or Coupling, cyclization, reduction, and Michael addition to afford Piperazine 24 in Org Proc Res Dev., 2011, 15(6). 1328-1335. Compounds (3a), (4a), and (5a) can be prepared as described in Scheme 1.

tert-Butyl (S)-9-bromo-10-fluoro-12-oxo-1,2,4,4a,5,6-hexahydro-3H,12H-benzo[b]pyrazino[1,2-e][1,5]oxazocine-3-carboxylate is synthesized using a 7-step sequence beginning with commercially available S-aspartic acid, which is the source of the stereocenter.

It is known that significant impurity rejection including rejection of the R-enantiomer can be achieved in the isolation of intermediates. tert-Butyl (S)-9-bromo-10-fluoro-12-oxo-1,2,4,4a,5,6-hexahydro-3H,12H-benzo[b]pyrazino[1,2-e][1,5]oxazocine-3-carboxylate can be isolated by crystallization with significant impurity rejection. Steps and Schemes are used to control process and reagent impurities.

The present disclosure provides a method of preparation of an intermediate compound of the Formula iIIa:

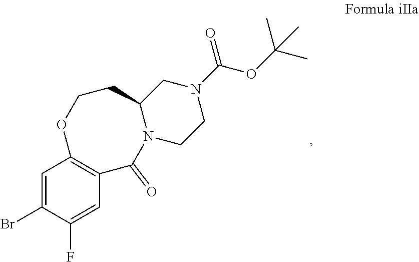 [SMILES: CC(C)(C)OC(=O)N1CCN2C(=O)c3cc(F)c(Br)cc3OCC[C@H]2C1]

or a pharmaceutically acceptable salt thereof, comprising the step of:
cyclization of an intermediate compound of the Formula II:

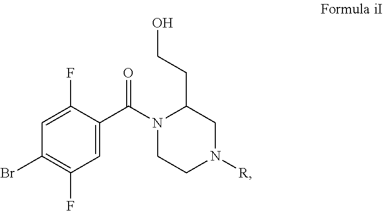

or of the Formula iIa:

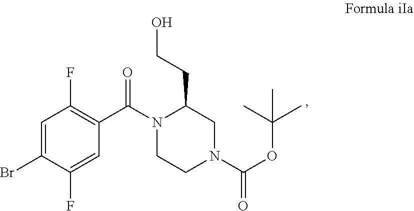 [SMILES: CC(C)(C)OC(=O)N1CCN(C(=O)c2cc(F)c(Br)cc2F)[C@@H](CCO)C1]

or a pharmaceutically acceptable salt thereof, by use of a cyclization base.

In an embodiment, the cyclization base is selected from the group consisting of sodium hydride, N,N-diisopropylethylamine (DIPEA or DIEA), triethylamine (TEA), cesium carbonate, diazabicycloundecene (DBU), sodium tert-butoxide, sodium tert-pentoxide, sodium tert-amylate, potassium tert-pentoxide, and potassium tert-butoxide.

In another embodiment, the method of preparation further comprises a cyclization solvent. In an embodiment the cyclization solvent is N,N-dimethylformamide (DMF).

The method of preparation wherein the step of cyclization is conducted at about 0° C.

Certain stereochemical centers have been left unspecified and certain substituents have been eliminated in the following schemes for the sake of clarity and are not intended to limit the teaching of the schemes in any way. It will be understood that all stereoisomers are encompassed. Specific stereoisomers can be prepared by stereospecific synthesis using enantiomerically pure or enriched starting materials and/or reagents. Alternatively, enantiomers can be separated using methods known in the art, such as chiral chromatography or by converting the enantiomers to diastereomeric salts, separating the diastereomeric salts, converting the diastereomeric salt into a non-salt form and isolating the enantiomer.

Individual isomers, enantiomers, and diastereomers may be separated or resolved by one of ordinary skill in the art at any convenient point in the synthesis of compounds of the disclosure, by methods such as selective crystallization techniques or chiral chromatography (See for example, J. Jacques, et al., “Enantiomers, Racemates, and Resolutions”, John Wiley and Sons, Inc., 1981, and E. L. Eliel and S. H. Wilen, “Stereochemistry of Organic Compounds”, Wiley-Interscience, 1994).

Certain abbreviations are defined as follows: “ACN” refers to acetonitrile; “APCI-MS” refers to atmospheric pressure chemical ionization mass spectrometry; “Boc” refers to tert-butoxycarbonyl; “CDI” refers 1,1′-carbonyldiimidazole; “CDMT” refers to 2-chloro-4,6-dimethoxy-1,3,5-triazine; “DCC” refers to 1,3-dicyclohexylcarbodiimide; “DCM” refers to dichloromethane; “DIC” refers to 1,3-diisopropylcarbodiimide; “DIPEA” or “DIEA” refers to N,N-diisopropylethylamine; “DMAc” or “DMA” refer to dimethylacetamide; “DMAP” refers to 4-dimethylaminopyridine; “DMF” refers to N,N-dimethylformamide; “DMSO” refers to dimethylsulfoxide; “DMSO-d6” refers to deuterated dimethylsulfoxide; “EDCI” refers to 1-(3-dimethylaminopropyl)-3-ethylcarbodiimide hydrochloride; “EtOAc” refers to ethyl acetate; “EtOH” refers to ethanol or ethyl alcohol; “HATU” refers to O-(7-Azabenzotriazol-1-yl)-N,N,N′,N′-tetramethyluronium hexafluorophosphate; “HOAt” refers to 1-hydroxy-7-azobenzotriazole; “HBTU” refers to 3-[bis(dimethylamino)methyliumyl]-3H-benzotriazol-1-oxide hexafluorophosphate; “HOBt” refers to 1-hydroxylbenzotriazole hydrate; “HPLC” refers to high-performance liquid chromatography; “MeOH” refers to methanol; “2-MeTHF” refers to 2-methyltetrahydrofuran; “MP (DSC)” refers to melting point by differential scanning calorimetry; “MTBE” refers to methyl tert-butyl ether; “NCS” refers to N-chlorosuccinimide; “NMM” refers to N-methylmorpholine; “NMP” refers to N-methyl-2-pyrrolidone; “NMR” refers to nuclear magnetic resonance; “PG” refers to protecting group; “PyBOP” refers to (benzotriazol-1-yl-oxytripyrrolidinophosphoniuin hexafluorophosphate); “PyBrOP” refers to bromo(tri-pyrrolidinyl)phosphoniumhexafluorophosphate; and “TEA” refers to triethylamine; “TFA” refers to trifluoroacetic acid; and “THF” refers to tetrahydrofuran.

A “cyclization base” is selected from the group consisting of sodium hydride, N,N-diisopropylethylamine (DIPEA or DIEA), triethylamine (TEA), cesium carbonate, diazabicycloundecene (DBU), sodium tert-butoxide, sodium tert-pentoxide, sodium tert-amylate, potassium tert-pentoxide, and potassium tert-butoxide.

### PREPARATIONS AND EXAMPLES

### Preparation 1

### Methyl 4-amino-3-chloro-2,5-difluoro-benzoate

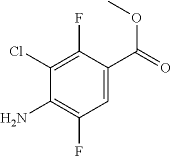

The title compound was prepared in the same manner as the method of WO2021/118877; Preparation 31. ES/MS m/z: 222 [M+H]⁺.

### Preparation 2

### Methyl 3-chloro-2,5-difluoro-4-iodo-benzoate

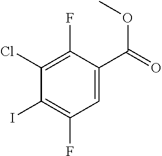

Cuprous iodide (22.7 g, 119 mmol), ACN (100 mL) and tert-butyl nitrite (17.7 mL, 149 mmol) were combined and stirred for 45 minutes at room temperature. A solution of methyl 4-amino-3-chloro-2,5-difluoro-benzoate (22.1 g, 100 mmol) in ACN (100 mL) was added. The reaction mixture was stirred at 40° C. for three hours, allowed to cool to room temperature, filtered through diatomaceous earth, and rinsed with DCM. The filtrate was concentrated in vacuo and purified by silica gel flash chromatography (10% EtOAc in hexanes) to give the title compound as a white solid (17.8 g, 54%). ¹H NMR (CDCl₃) δ 7.60 (dd, J=5.6, 7.6 Hz, 1H), 3.98 (s, 3H).

### Preparation 3

### 3-Chloro-2,5-difluoro-4-iodo-benzoic acid

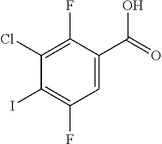 [SMILES: O=C(O)c1cc(F)c(I)c(Cl)c1F]

MeOH (50 mL) was added to a solution of methyl 3-chloro-2,5-difluoro-4-iodo-benzoate (16.8 g, 50.5 mmol) in THE (125 mL). The reaction mixture was cooled in an ice bath. 1N aqueous NaOH (150 mL, 150 mmol) was added in three portions. The reaction mixture was allowed to stir at room temperature for 30 minutes and concentrated in vacuo to remove organic solvents. The aqueous remainder was cooled with ice chips and the pH adjusted to ˜2 with 5N aqueous HCl (25 mL). Extracted with EtOAc (3×). The combined organic extracts were dried over sodium sulfate, filtered, and concentrated in vacuo to give the title compound (15.8 g, 98%). ¹H NMR 6 (DMSO-d6) 13.91 (s, 1H), 7.68 (dd, J=5.8, 7.8 Hz, 1H).

### Preparation 4

### tert-Butyl (3R)-4-(3-chloro-2,5-difluoro-4-iodo-benzoyl)-3-(hydroxymethyl)piperazine-1-carboxylate

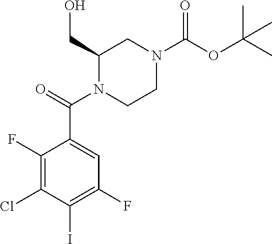 [SMILES: CC(C)(C)OC(=O)N1CCN(C(=O)c2cc(F)c(I)c(Cl)c2F)[C@@H](CO)C1]

HATU (3.31 g, 8.53 mmol) was added to a solution of 3-chloro-2,5-difluoro-4-iodo-benzoic acid (2.99 g, 9.39 mmol) and DIEA (6.7 mL, 38 mmol) in THE (100 mL). The reaction mixture was stirred at room temperature for one hour. A solution of tert-butyl (3R)-3-(hydroxymethyl)piperazine-1-carboxylate (1.92 g, 8.52 mmol) in THE (5 mL) was added dropwise. The reaction mixture was stirred at room temperature for 14 hours and 60° C. for three hours, then diluted with EtOAc and water. The organic layer was washed with water, 1N aqueous NaOH, and brine. The aqueous layer was extracted with EtOAc, which was washed with brine. The combined organic extracts were dried over sodium sulfate, filtered, and concentrated in vacuo. The crude was purified by silica gel flash chromatography (10-50% EtOAc in hexanes) to give the title compound as a white solid (3.82 g, 82%). ES/MS m/z (³⁵Cl/³⁷Cl) 461/463 [M-tert-Butyl+H]⁺.

### Preparation 5

### tert-Butyl (13aS)-9-bromo-10-chloro-8-fluoro-6-oxo-1,3,4,12,13,13a-hexahydropyrazino[2,1-d][1,5]benzoxazocine-2-carboxylate

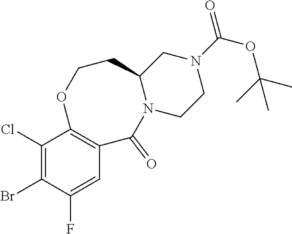 [SMILES: CC(C)(C)OC(=O)N1CCN2C(=O)c3cc(F)c(Br)c(Cl)c3OCC[C@H]2C1]

The title compound was prepared in the same manner as the method of WO2021/118877; Preparation 117. ES/MS m/z (⁷⁹Br/⁸¹Br) 407/409 [M-tert-Butyl+H]⁺.

### Preparation 6

### tert-Butyl (4aR)-7-chloro-9-fluoro-8-iodo-11-oxo-2,4,4a,5-tetrahydro-1H-pyrazino[2,1-c][1,4]benzoxazepine-3-carboxylate

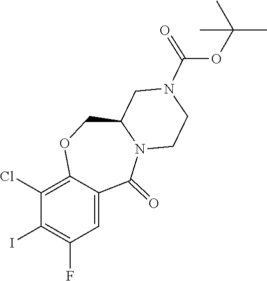 [SMILES: CC(C)(C)OC(=O)N1CCN2C(=O)c3cc(F)c(I)c(Cl)c3OC[C@H]2C1]

tert-Butyl (3R)-4-(3-chloro-2,5-difluoro-4-iodo-benzoyl)-3-(hydroxymethyl)piperazine-1-carboxylate (4.59 g, 8.88 mmol) in DMF (150 mL) was cooled to 0° C. Sodium hydride (0.89 g, 22 mmol, 60 mass % in paraffin oil) was added. After four hours at 0° C., the reaction mixture was quenched with saturated aqueous sodium bicarbonate and diluted with water and diethyl ether. The aqueous layer was extracted with diethyl ether (4×). The combined organic extracts were washed with water (3×) and brine, dried over sodium sulfate, filtered, and concentrated in vacuo. The crude was purified by silica gel flash chromatography (15-40% EtOAc in hexanes) to give the title compound as a white solid (3.82 g, 82%). ES/MS m/z (³⁵Cl/³⁷Cl) 441/443 [M-tert-Butyl+H]⁺.

### Preparation 7

### tert-Butyl N-(4-bromo-3-cyano-7-fluoro-benzothiophen-2-yl)carbamate

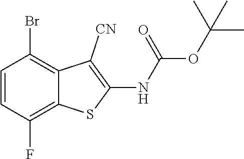 [SMILES: CC(C)(C)OC(=O)Nc1sc2c(F)ccc(Br)c2c1C#N]

The title compound was prepared in the same manner as the method of WO2021/118877; Preparation 14. ES/MS m/z (⁷⁹Br/⁸¹Br) 315/317 [M-tert-Butyl+H]⁺.

### Preparation 8

### tert-Butyl N-[3-cyano-7-fluoro-4-(4,4,5,5-tetramethyl-1,3,2-dioxaborolan-2-yl)benzothiophen-2-yl]carbamate

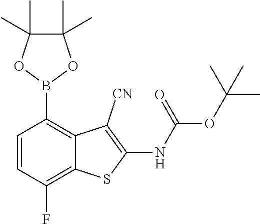 [SMILES: CC(C)(C)OC(=O)Nc1sc2c(F)ccc(B3OC(C)(C)C(C)(C)O3)c2c1C#N]

A mixture of tert-butyl N-(4-bromo-3-cyano-7-fluoro-benzothiophen-2-yl)carbamate (3.10 g, 8.35 mmol), bis(pinacolato)diboron (21.0 g, 82.7 mmol), and potassium acetate (2.50 g, 25.5 mmol) in 1,4-dioxane (50 mL) was flushed with nitrogen (direct sparge) for 10 minutes. 1,1′-Bis(diphenylphosphino)ferrocene-palladium(II)dichloride dichloromethane complex (1.0 g, 1.2 mmol) was added. The reaction flask was sealed and heated at 80-85° C. for 3.5 hours. The reaction mixture was filtered through diatomaceous earth. The filtrate was diluted with water and extracted with EtOAc (2×). The combined organic extracts were washed with brine, dried over sodium sulfate, filtered, and concentrated in vacuo. The crude was purified by silica gel flash chromatography (10-50% (20% acetone in DCM) in hexanes) to give the title compound (4.45 g, 87%). ES/MS m/z 363 [M-tert-Butyl+H]⁺.

### Preparation 9

### tert-Butyl N-[3-cyano-4-(5,5-dimethyl-1,3,2-dioxaborinan-2-yl)-7-fluoro-benzothiophen-2-yl]carbamate

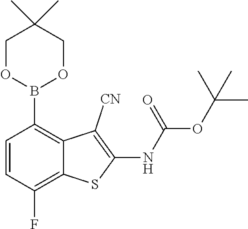 [SMILES: CC1(C)COB(c2ccc(F)c3sc(NC(=O)OC(C)(C)C)c(C#N)c23)OC1]

The title compound was prepared in the same manner as the method of WO2021/118877; Preparation 15. ¹H NMR (DMSO-d6) δ 11.6 (s, 1H), 7.61 (m, 1H), 7.20 (m, 1H), 3.78 (s, 4H), 1.54 (s, 9H), 1.03 (s, 6H).

### Preparations 10 and 11

### tert-Butyl (13aS)-9-[2-(tert-butoxycarbonylamino)-3-cyano-benzothiophen-4-yl]-10-chloro-8-fluoro-6-oxo-1,3,4,12,13,13a-hexahydropyrazino[2,1-d][1,5]benzoxazocine-2-carboxylate, P and M Atropisomers

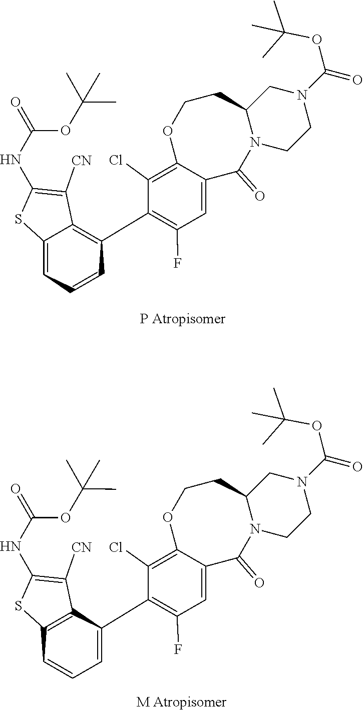

The title compounds were prepared in the same manner as the method of WO2021/118877; Preparation 167. The mixture of atropisomers were separated by silica gel flash chromatography (0-30% acetone in hexanes). Preparation 10 (P Atropisomer) was the first compound to elute off the column. Preparation 11 (M Atropisomer) was the second compound to elute off the column. For both, ES/MS m/z (³⁵Cl/³⁷Cl) 657/659 [M+H]⁺.

### Preparations 12 and 13

### tert-Butyl (13aS)-9-[2-(tert-butoxycarbonylamino)-3-cyano-7-fluoro-benzothiophen-4-yl]-10-chloro-8-fluoro-6-oxo-1,3,4,12,13,13a-hexahydropyrazino[2,1-d][1,5]benzoxazocine-2-carboxylate, P and M Atropisomers

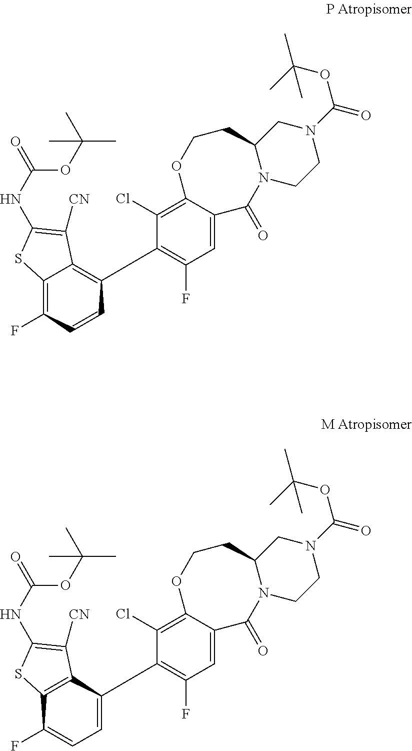

A mixture of tert-butyl (13aS)-9-bromo-10-chloro-8-fluoro-6-oxo-1,3,4,12,13,13a-hexahydropyrazino[2,1-d][1,5]benzoxazocine-2-carboxylate (10.0 g, 21.6 mmol), tert-butyl N-[3-cyano-4-(5,5-dimethyl-1,3,2-dioxaborinan-2-yl)-7-fluoro-benzothiophen-2-yl]carbamate (12.3 g, 30.4 mmol), potassium carbonate (8.94 g, 64.7 mmol), and (S)-(−)-2,2′-bis(diphenylphosphino)-1,1′-binaphthyl palladium dichloride (3.84 g, 4.42 mmol) was added to 1,4-dioxane (215 mL) that had been flushed with nitrogen (direct sparge) for 30 minutes. The reaction flask was sealed and heated at 105° C. for 14 hours. Additional tert-butyl N-[3-cyano-4-(5,5-dimethyl-1,3,2-dioxaborinan-2-yl)-7-fluoro-benzothiophen-2-yl]carbamate (3.54 g, 8.75 mmol) and (S)-(−)-2,2′-bis(diphenylphosphino)-1,1′-binaphthyl palladium dichloride (0.97 g, 1.12 mmol) were added. The reaction flask was sealed and heated at 105° C. for 14 hours.

The reaction mixture was filtered through diatomaceous earth, and rinsed with EtOAc. The filtrate was concentrated in vacuo and diluted with EtOAc, water, and brine. The aqueous layer was extracted with EtOAc. The combined organic extracts were dried over magnesium sulfate, filtered, and concentrated in vacuo. The crude was purified by silica gel flash chromatography (10-40% acetone in hexanes). Preparation 12 (P Atropisomer) eluted first, followed by Preparation 13 (M Atropisomer). Impure fractions of P atropisomer were further purified by silica gel flash chromatography. This gave the two title compounds (P atropisomer, 0.35 g, 2%; M atropisomer, 5.6 g, 38%). For both, ES/MS m/z 619 [M-tert-Butyl+H]⁺.

### Preparations 14 and 15

### tert-Butyl (4aR)-8-[2-(tert-butoxycarbonylamino)-3-cyano-7-fluoro-benzothiophen-4-yl]-7-chloro-9-fluoro-11-oxo-2,4,4a,5-tetrahydro-1H-pyrazino[2,1-c][1,4]benzoxazepine-3-carboxylate, P and M Atropisomers

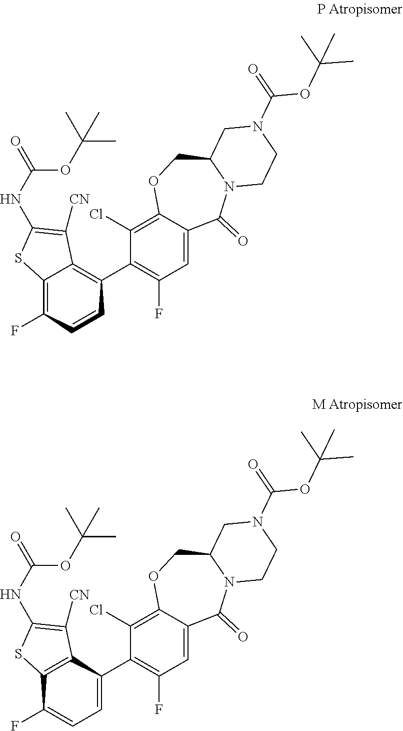

A solution of tert-butyl (4aR)-7-chloro-9-fluoro-8-iodo-11-oxo-2,4,4a,5-tetrahydro-1H-pyrazino[2,1-c][1,4]benzoxazepine-3-carboxylate (0.50 g, 1.01 mmol), tert-butyl N-[3-cyano-7-fluoro-4-(4,4,5,5-tetramethyl-1,3,2-dioxaborolan-2-yl)benzothiophen-2-yl]carbamate (0.63 g, 1.51 mmol), and potassium phosphate (0.50 g, 2.36 mmol) in water (2 mL) and 1,4-dioxane (10 mL) was flushed with nitrogen (direct sparge) for 10 minutes. 1,1′-Bis(di-tert-butylphosphino)ferrocene palladium dichloride (0.20 g, 0.30 mmol) was added. The reaction flask was sealed and heated at 70° C. for three hours.

A solution of tert-butyl (4aR)-7-chloro-9-fluoro-8-iodo-11-oxo-2,4,4a,5-tetrahydro-1H-pyrazino[2,1-c][1,4]benzoxazepine-3-carboxylate (0.20 g, 0.40 mmol), tert-butyl N-[3-cyano-7-fluoro-4-(4,4,5,5-tetramethyl-1,3,2-dioxaborolan-2-yl)benzothiophen-2-yl]carbamate (0.24 g, 0.57 mmol), and potassium phosphate (0.20 g, 0.96 mmol) in water (2.5 mL) and 1,4-dioxane (8 mL) was flushed with nitrogen (direct sparge) for 10 minutes. 1,1′-Bis(di-tert-butylphosphino)ferrocene palladium dichloride (0.080 g, 0.12 mmol) was added. The reaction flask was sealed and heated at 70° C. for five hours.

The two reaction mixtures were combined, filtered through diatomaceous earth, and rinsed with EtOAc. The filtrate was diluted with MTBE and saturated aqueous sodium bicarbonate. The organic extract was washed with brine, dried over magnesium sulfate, filtered, and concentrated in vacuo. The crude was purified by silica gel flash chromatography (0-30% acetone in hexanes). Preparation 14 (P Atropisomer) eluted first, followed by Preparation 15 (M Atropisomer). Impure fractions of P atropisomer were further purified by silica gel flash chromatography (0-100% EtOAc in hexanes). This gave the two title compounds (P atropisomer, 0.17 g, 17%; M atropisomer, 0.21 g, 21%). For both, ES/MS m/z (³⁵Cl/³⁷Cl) 605/607 [M-tert-Butyl+H]⁺.

### Preparation 16

### 4-[(13aS)-10-Chloro-8-fluoro-6-oxo-2,3,4,12,13,13a-hexahydro-1H-pyrazino[2,1-d][1,5]benzoxazocin-9-yl]-2-amino-benzothiophene-3-carbonitrile, P Atropisomer

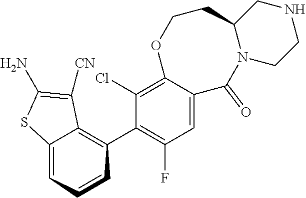

TFA (4 mL) was added to a solution of tert-butyl (13aS)-9-[2-(tert-butoxycarbonylamino)-3-cyano-benzothiophen-4-yl]-10-chloro-8-fluoro-6-oxo-1,3,4,12,13,13a-hexahydropyrazino[2,1-d][1,5]benzoxazocine-2-carboxylate, P Atropisomer (0.98 g, 1.5 mmol) in DCM (5 mL). The reaction mixture was stirred at room temperature for three hours, concentrated in vacuo, and purified by silica gel flash chromatography (4-10% 7N ammoniated MeOH in DCM) to give the title compound (0.57 g, 84%). ES/MS m/z (³⁵Cl/³⁷Cl) 457/459 [M+H]⁺.

### Preparation 17

### 4-[(13aS)-10-Chloro-8-fluoro-6-oxo-2,3,4,12,13,13a-hexahydro-1H-pyrazino[2,1-d][1,5]benzoxazocin-9-yl]-2-amino-benzothiophene-3-carbonitrile, M Atropisomer

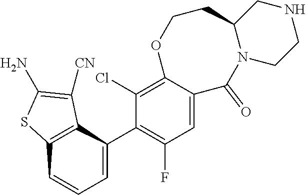

The title compound was prepared in the same manner as the method of WO2021/118877; Preparation 187. ES/MS m/z (³⁵Cl/³⁷Cl) 457/459 [M+H]⁺.

### Preparation 18

### 4-[(4aR)-7-Chloro-9-fluoro-11-oxo-1,2,3,4,4a,5-hexahydropyrazino[2,1-c][1,4]benzoxazepin-8-yl]-2-amino-7-fluoro-benzothiophene-3-carbonitrile, P Atropisomer

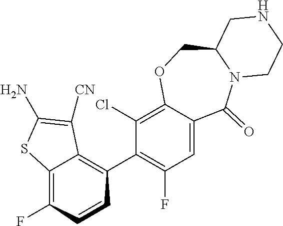

TFA (2 mL) was added to a solution of tert-butyl (4aR)-8-[2-(tert-butoxycarbonylamino)-3-cyano-7-fluoro-benzothiophen-4-yl]-7-chloro-9-fluoro-11-oxo-2,4,4a,5-tetrahydro-1H-pyrazino[2,1-c][1,4]benzoxazepine-3-carboxylate, P Atropisomer (0.018 g, 0.027 mmol) in DCM (2 mL). The reaction mixture was stirred at room temperature for 30 minutes, concentrated in vacuo, and purified by silica gel flash chromatography (0-10% MeOH in DCM, then 0-10% 7N ammoniated MeOH in DCM) to give the title compound (0.013 g, 100%). ES/MS m/z (³⁵Cl/³⁷Cl) 461/463 [M+H]⁺.

### Preparation 19

### 4-[(4aR)-7-Chloro-9-fluoro-11-oxo-1,2,3,4,4a,5-hexahydropyrazino[2,1-c][1,4]benzoxazepin-8-yl]-2-amino-7-fluoro-benzothiophene-3-carbonitrile, M Atropisomer

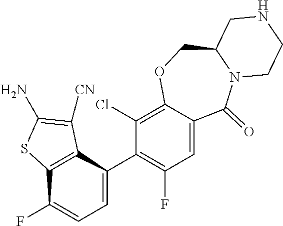

TFA (2.5 mL) was added to an ice-cooled solution of tert-butyl (4aR)-8-[2-(tert-butoxycarbonylamino)-3-cyano-7-fluoro-benzothiophen-4-yl]-7-chloro-9-fluoro-11-oxo-2,4,4a,5-tetrahydro-1H-pyrazino[2,1-c][1,4]benzoxazepine-3-carboxylate, M Atropisomer (0.20 g, 0.31 mmol) in DCM (2.5 mL). The reaction mixture was stirred at room temperature for two hours, concentrated in vacuo, and purified by silica gel flash chromatography (0-10% 7N ammoniated MeOH in DCM) to give the title compound (0.13 g, 89%). ES/MS m/z (³⁵Cl/³⁷Cl) 461/463 [M+H]⁺.

### Examples 1 and 2

### (13aS)-9-(2-Amino-7-fluoro-1,3-benzothiazol-4-yl)-8,10-dichloro-2-prop-2-enoyl-1,3,4,12,13,13a-hexahydropyrazino[2,1-d][1,5]benzoxazocin-6-one, P and M Atropisomers

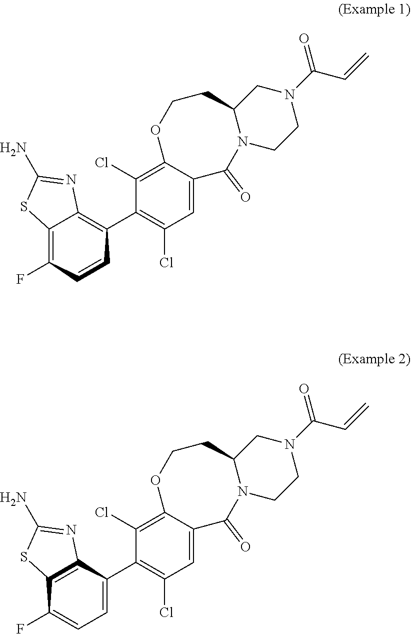

The title compounds were prepared in the same manner as the method of WO2021/118877; Example 1. The mixture of atropisomers was separated using Chiralpak® IC, 4.6×150 mm, 40% EtOH/CO₂, 5 mL/min, 225 nm. Example 1 (P Atropisomer) is the first compound off the column. Example 2 (M Atropisomer) is the second compound off the column. For both, ES/MS m/z (³⁵Cl/³⁷Cl) 521/523 [M+H]⁺.

### Example 3

### 4-[(13aS)-10-Chloro-8-fluoro-6-oxo-2-prop-2-enoyl-1,3,4,12,13,13a-hexahydropyrazino[2,1-d][1,5]benzoxazocin-9-yl]-2-amino-benzothiophene-3-carbonitrile, P Atropisomer

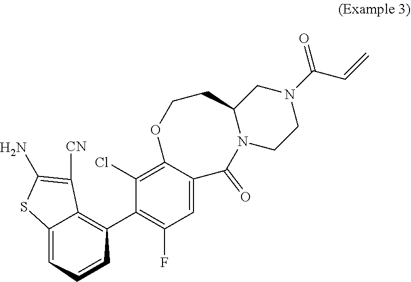

A solution of 4-[(13aS)-10-Chloro-8-fluoro-6-oxo-2,3,4,12,13,13a-hexahydro-1H-pyrazino[2,1-d][1,5]benzoxazocin-9-yl]-2-amino-benzothiophene-3-carbonitrile, P Atropisomer (0.115 g, 0.252 mmol) and DIEA (0.20 mL, 1.2 mmol) in DCM (2 mL) was cooled to −78° C. Acryloyl chloride (0.02 mL, 0.2 mmol) was added. After five minutes, diluted with a small amount of 2-propanol and concentrated in vacuo. The crude was purified by silica gel flash chromatography (20-100% acetone in hexanes) to give the title compound (0.091 g, 71%). ES/MS m/z (³⁵Cl/³⁷Cl) 511/513 [M+H]⁺.

### Example 4

### 4-[(13aS)-10-Chloro-8-fluoro-6-oxo-2-prop-2-enoyl-1,3,4,12,13,13a-hexahydropyrazino[2,1-d][1,5]benzoxazocin-9-yl]-2-amino-benzothiophene-3-carbonitrile, M Atropisomer

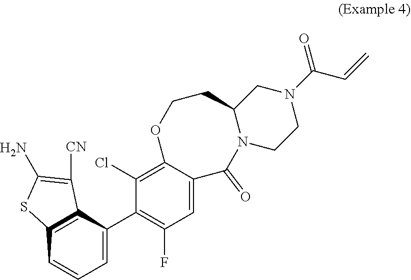

The title compound was prepared in the same manner as the method of WO2021/118877; Example 34. ES/MS m/z (³⁵Cl/³⁷Cl) 511/513 [M+H]⁺.

### Example 5

### 4-[(13aS)-10-Chloro-8-fluoro-6-oxo-2-prop-2-enoyl-1,3,4,12,13,13a-hexahydropyrazino[2,1-d][1,5]benzoxazocin-9-yl]-2-amino-7-fluoro-benzothiophene-3-carbonitrile, P Atropisomer

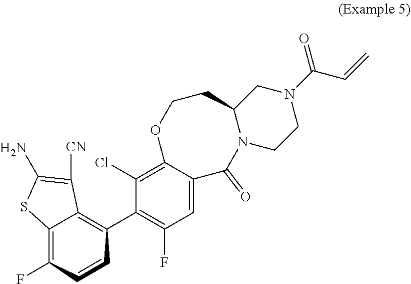

TFA (1 mL) was added to a solution of tert-butyl (13aS)-9-[2-(tert-butoxycarbonylamino)-3-cyano-7-fluoro-benzothiophen-4-yl]-10-chloro-8-fluoro-6-oxo-1,3,4,12,13,13a-hexahydropyrazino[2,1-d][1,5]benzoxazocine-2-carboxylate, P Atropisomer (0.025 g, 0.037 mmol) in DCM (1 mL). The reaction mixture was stirred at room temperature for 30 minutes, then concentrated in vacuo to give crude deprotected material.

The residue was dissolved in DCM (1 mL) and DIEA (0.013 mL, 0.074 mmol) was added. The solution was cooled to −78° C. Acryloyl chloride (0.0030 mL) in DCM (1 mL) was added. After 30 minutes, concentrated in vacuo. The crude was purified by silica gel flash chromatography (20-80% acetone in hexanes) to give the title compound (0.012 g, 61%). ES/MS m/z (³⁵Cl/³⁷Cl) 529/531 [M+H]⁺.

### Example 6

### 4-[(13aS)-10-Chloro-8-fluoro-6-oxo-2-prop-2-enoyl-1,3,4,12,13,13a-hexahydropyrazino[2,1-d][1,5]benzoxazocin-9-yl]-2-amino-7-fluoro-benzothiophene-3-carbonitrile, M Atropisomer (“KRAS G12C Inh”)

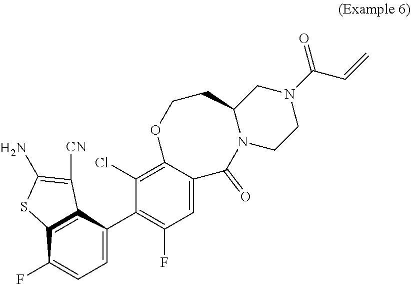

HCl gas was bubbled for five minutes into an ice-cooled solution of tert-butyl (13aS)-9-[2-(tert-butoxycarbonylamino)-3-cyano-7-fluoro-benzothiophen-4-yl]-10-chloro-8-fluoro-6-oxo-1,3,4,12,13,13a-hexahydropyrazino[2,1-d][1,5]benzoxazocine-2-carboxylate, M Atropisomer (0.786 g, 1.17 mmol) in DCM (12 mL) and 2-propanol (12 mL). The reaction mixture was stirred at room temperature for five hours, then cooled in an ice bath. HCl gas was bubbled into the reaction mixture for five minutes. The reaction mixture was stirred at room temperature for 14 hours, then concentrated in vacuo. The residue was twice diluted with n-heptane and concentrated in vacuo. MTBE (50 mL) was added. The mixture was stirred at room temperature for ten minutes, then filtered to give the deprotected material as a dihydrochloride salt.

The dihydrochloride salt was dissolved in water (12 mL). 2-Methyltetrahydrofuran (12 mL) was added. A solution of potassium carbonate (0.81 g, 5.82 mmol) in water (12 mL) was added. The mixture was vigorously stirred while being cooled in an ice bath. Acryloyl chloride (0.10 mL, 1.28 mmol) in 2-methyltetrahydrofuran (5 mL) was added dropwise. After five minutes, diluted with brine and 2-methyltetrahydrofuran. The aqueous layer was extracted with 2-methyltetrahydrofuran (2×). The combined organic extracts were washed with water, dried over magnesium sulfate, filtered, and concentrated in vacuo. The crude was purified by silica gel flash chromatography (20-30% B in A; A:3:1 EtOAc:hexanes; B: 4:1 EtOAc:MeOH) to give the title compound (0.409 g, 66%). ES/MS m/z (³⁵Cl/³⁷Cl) 529/531 [M+H]⁺.

### Example 7

### 4-[(4aR)-7-Chloro-9-fluoro-11-oxo-3-prop-2-enoyl-2,4,4a,5-tetrahydro-1H-pyrazino[2,1-c][1,4]benzoxazepin-8-yl]-2-amino-7-fluoro-benzothiophene-3-carbonitrile, P Atropisomer

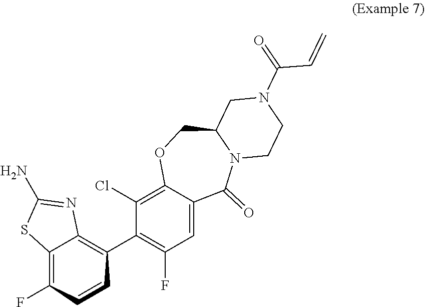

Acryloyl chloride (0.0022 mL, 0.027 mmol) was added to an ice-cooled mixture of 4-[(4aR)-7-chloro-9-fluoro-11-oxo-1,2,3,4,4a,5-hexahydropyrazino[2,1-c][1,4]benzoxazepin-8-yl]-2-amino-7-fluoro-benzothiophene-3-carbonitrile, P Atropisomer (0.013 g, 0.027 mmol) and potassium carbonate (0.011 g, 0.082 mmol) in EtOAc (0.27 mL) and water (0.27 mL). After 20 minutes, diluted with EtOAc and brine. The organic extract was dried over sodium sulfate, filtered, and concentrated in vacuo. The crude was purified by silica gel flash chromatography (20-100% acetone in hexanes) to give the title compound (0.011 g, 78%). ES/MS m/z (³⁵Cl/³⁷Cl) 515/517 [M+H]⁺.

### Example 8

### 4-[(4aR)-7-Chloro-9-fluoro-11-oxo-3-prop-2-enoyl-2,4,4a,5-tetrahydro-1H-pyrazino[2,1-c][1,4]benzoxazepin-8-yl]-2-amino-7-fluoro-benzothiophene-3-carbonitrile, M Atropisomer

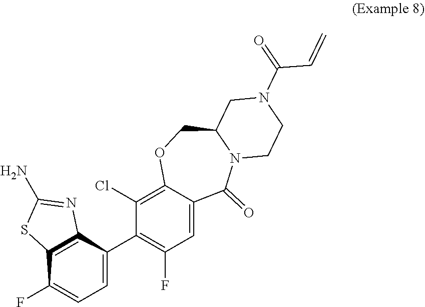

Acryloyl chloride (0.25M in DCM, 1.1 mL, 0.28 mmol) was added to an ice-cooled mixture of 4-[(4aR)-7-chloro-9-fluoro-11-oxo-1,2,3,4,4a,5-hexahydropyrazino[2,1-c][1,4]benzoxazepin-8-yl]-2-amino-7-fluoro-benzothiophene-3-carbonitrile, M Atropisomer (0.128 g, 0.28 mmol) and potassium carbonate (0.12 g, 0.83 mmol) in EtOAc (3 mL) and water (3 mL). After five minutes, diluted with EtOAc and separated layers. The organic extract was dried over magnesium sulfate, filtered, and concentrated in vacuo. The crude was purified by silica gel flash chromatography (0-10% 7N ammoniated MeOH in DCM) to give the title compound (0.13 g, 87%). ES/MS m/z (³⁵Cl/³⁷Cl) 515/517 [M+H]⁺.

### Biological Assays

The following assays demonstrate that the combination of compounds described herein are inhibitors of KRas G12C and inhibit growth of certain tumors in vitro and/or in vivo.

### Example 1: Protocol for KRAS G12C Inh in Combination with Aurora A Inh

The purpose of these studies was to evaluate the anti-tumor growth activities of KRAS G12C Inh in combination with the Aurora A Inh in human NSCLC tumor xenograft or PDX models harboring a KRAS G12C mutation. Synergy or additivity was observed for KRAS G12C Inh in combination with Aurora A Inh in cell proliferation assays. In animal models, KRAS G12C Inh demonstrated dose- and time-dependent inhibition of KRAS activity and downstream signaling and led to significant tumor growth inhibition/regression in a panel of KRAS G12C mutant NSCLC, CRC, and PDAC xenograft and PDX models. In addition, KRAS G12C Inh showed additive effects in KRAS G12C mutant in vivo models when combined with Aurora A Inh.

KRAS G12C Inh also demonstrated different degrees of antitumor activity in KRAS G12C mutant animal models. In order to achieve a more durable response and in a broader range of tumors, combining KRAS G12C Inh with other therapies could provide superior clinical activity. KRAS G12C Inh combinations were evaluated in multiple KRAS G12C mutant in vivo models including 2 lung cancer xenograft models (H358 and H1373), 1 lung cancer PDX model (EL3187), and 1 CRC xenograft model (SW837). KRAS G12C Inh alone at suboptimal doses and monotherapy of Aurora A Inh showed various antitumor activities in these models. The combination of KRAS G12C Inh Aurora A Inh led to improved antitumor activity, based on tumor growth inhibition and tumor regression. This combination demonstrated increased efficacy over monotherapy and no adverse effects on body weight.

For H358 and H1373 xenograft tumor growth, 5×10⁶ cells in 0.2 mL Hanks' Balanced Salt solution (HBSS):Matrigel (Corning, Cat #354234) (1:1) were implanted subcutaneously in the right flank of each animal. H358 cells were implanted into NOD SCID gamma mice (20-22 g, The Jackson Laboratory, Bar Harbor, Maine) and H1373 cells were implanted into female athymic nude mice (20-22 g, Envigo RMS, Inc., Mount Comfort, Indiana). Tumor volumes were measured using calipers twice weekly. When the tumor volumes reached 200-300 mm³, the mice were randomized (n=5 or 6 per group) based on tumor measurement and body weight using the multi-task block randomization tool. Treatment was initiated with oral administration (gavage) of 0.2 mL vehicle (10% N-Methyl-2-pyrrolidone “NMP”/90% 15% w/v Polyvinylpyrrolidone-vinyl acetate “PVP-VA” in PEG 400), KRAS G12C Inh (10% NMP/90% 15% w/v PVP-VA in PEG 400) at 10 mg/kg QD, Aurora A Inh (20% w/v 2-Hydroxypropyl-β-Cyclodextrin “HPBCD” in sterile water) at 57 mg/kg BID, or the combination of KRAS G12C Inh at 10 mg/kg QD and Aurora A Inh at 57 mg/kg BID for 28 days. In the H1373 model, starting on Day 4, Aurora A Inh dosing was changed to 57 mg/kg QD. Statistical analysis results were summarized at Day 28 of treatment for the H358 xenograft study and at Day 24 of treatment for the H1373 xenograft study.

For EL3187 PDX model, tumor fragments were subcutaneously implanted into the right rear flank of female athymic nude-Foxn1nu feeder mice (20-22 g, Envigo RMS, Inc., Mount Comfort, Indiana). Once tumor volumes reached 800-1000 mm³, the animals were sacrificed and the tumors were harvested using aseptic technique. Fresh tumors, passage 4, were cut into 10-15 mm³ fragments and placed into cold Gibco Hibernate Medium, and then the pooled tumor fragments were subcutaneously implanted into animals with a 10 g trochar needle. When the tumor volumes reached 200-300 mm³, the mice were randomized (n=5 per group). Treatment was initiated with oral administration (gavage) of 0.2 mL vehicle (10% NMP/90% 15% w/v PVP-VA in PEG 400), KRAS G12C Inh (10% NMP/90% 15% w/v PVP-VA in PEG 400) at 3 mg/kg QD, Aurora A Inh (20% w/v HPBCD in sterile water) at 57 mg/kg BID, or the combination of KRAS G12C Inh at 3 mg/kg QD and Aurora A Inh at 57 mg/kg BID for 22 days. Statistical analysis results were summarized at Day 22 of treatment.

TABLE 1
Tumor Growth Inhibition of KRAS G12C Inhibitor in Combination
with Aurora A Inhibitor in H358 Lung Xenograft Model

| | | Dose | Dose | % Delta | % Tumor | |
|---|---|---|---|---|---|---|
| Group | Agent | (mg/kg) | Frequency | T/C | Regression | n |
| 01 | KRAS G12C Inh | 10 | PO, QD × 28 | 25.1 | N/A | 5 |
| 02 | Aurora A Inh | 57 | PO, BID × 28 | 22.4 | N/A | 5 |
| 03 | KRAS G12C Inh | 10 | PO, QD × 28 | N/A | 55.3 | 4 |
| | Aurora A Inh | 57 | PO, BID × 28 | | | |

Table 1 shows data for the H358 NSCLC xenograft model compared single agent dosing of KRAS G12C Inh and single agent dosing of Aurora A Inh, versus the combination dosing of KRAS G12C Inh and Aurora A Inh. Treatment with KRAS G12C Inh in combination with Aurora A Inh demonstrated in vivo efficacy in the H358 NSCLC xenograft model relative to either single agent dosing and resulted in 55.3% tumor regression.

TABLE 2
Tumor Growth Inhibition of KRAS G12C Inhibitor in Combination
with Aurora A Inhibitor in H1373 Lung Xenograft Model

| | | Dose | Dose | % Delta | % Tumor | |
|---|---|---|---|---|---|---|
| Group | Agent | (mg/kg) | Frequency | T/C | Regression | n |
| 01 | KRAS G12C Inh | 10 | PO, QD × 24 | 1.5 | N/A | 5 |
| 02 | Aurora A Inh | 57 | PO, BID × 3 + | 77.0 | N/A | 5 |
| | | | QD × 21* | | | |
| 03 | KRAS G12C Inh | 10 | PO, QD × 24 | N/A | 25.3 | 5 |
| | Aurora A Inh | 57 | PO, BID × 3 + | | | |
| | | | QD × 21* | | | |

*Dosing schedule for Aurora A Inh changed from 57 mg/kg BID to 57 mg/kg QD on treatment Day 4 and for the remainer of the study.

Table 2 shows data for the H1373 NSCLC xenograft model compared single agent dosing of KRAS G12C Inh and single agent dosing of Aurora A Inh versus the combination dosing of KRAS G12C Inh with Aurora A Inh. Treatment with KRAS G12C Inh in combination with Aurora A Inh demonstrated in vivo efficacy in the H1373 NSCLC xenograft model relative to either single agent dosing and resulted in 25.3% tumor regression.

TABLE 3
Tumor Growth Inhibition of KRAS G12C Inhibitor in Combination
with Aurora A Inhibitor in EL3187 (Lung) PDX Model

| | | Dose | Dose | % Delta | % Tumor | |
|---|---|---|---|---|---|---|
| Group | Agent | (mg/kg) | Frequency | T/C | Regression | n |
| 01 | KRAS G12C Inh | 3 | PO, QD × 22 | 15.2 | N/A | 5 |
| 02 | Aurora A Inh | 57 | PO, BID × 22 | N/A | 37.1 | 5 |
| 03 | KRAS G12C Inh | 3 | PO, QD × 22 | N/A | 88.6 | 4 |
| | Aurora A Inh | 57 | PO, BID × 22 | | | |

Table 3 shows data for the EL3187 NSCLC PDX model comparing single agent dosing of KRAS G12C Inh and single agent dosing of Aurora A Inh, versus the combination dosing of KRAS G12C Inh with Aurora A Inh. Treatment with KRAS G12C Inh in combination with Aurora A Inh demonstrated significant in vivo efficacy in the EL3187 NSCLC PDX model relative to either of the single agent dosing and resulted in 88.6% tumor regression.

% Delta T/C is calculated when the endpoint tumor volume in a treated group is at or above baseline tumor volume. The formula is 100*(T−T₀)/(C−C₀), where T and C are mean endpoint tumor volumes in the treated or control group, respectively. T₀ and C₀ are mean baseline tumor volumes in those groups.

% Tumor Regression is calculated when the endpoint tumor volume in a treated group is below baseline tumor volume. The formula is 100*(T−T₀)/T₀, where T₀ is the mean baseline tumor volume for the treated group.

### Additional Embodiments

Embodiment 1. A method of treating a patient for cancer wherein one or more cells express KRas G12C mutant protein, comprising administering to a patient in need thereof, an effective amount of a compound of the formula:

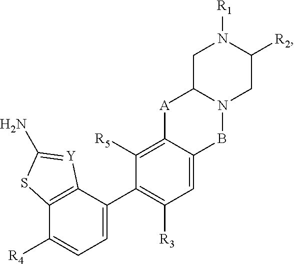

wherein:
A is —OCH₂—, —N(R₆)CH₂—, —OCH₂CH₂—, —N(R₆)CH₂CH₂—, —CH₂OCH₂—, or —CH₂N(R₆)CH₂—;
B is —CH₂— or —C(O)—;
Y is —C(CN)— or —N—;
R₁ is —CN, —C(O)C≡CR₈, or a group of the formula

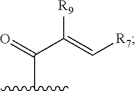

R₂ is H, methyl, or —CH₂CN;
R₃ and R₅ are each independently H, halogen, —C₀₋₃ alkyl-cyclopropyl, —C₁₋₆ alkyl optionally substituted 1-3 times with R₁₀, or —O—C₁₋₆ alkyl optionally substituted 1-3 times with R₁₀;
R₄ is H, halogen, or —C₁₋₆ alkyl optionally substituted 1-3 times with R₁₀;
R₆ is H or —C₁₋₆ alkyl optionally substituted 1-3 times with R₁₀;
R₇ is H, halogen, —NR₁₁R₁₂, —CH₂NR₁₁R₁₂, —C₁₋₆ alkyl optionally substituted 1-3 times with
R₁₀ or R₁₃, —C₀₋₃ alkyl cyclopropyl, or —O—C₁₋₆ alkyl optionally substituted 1-3 times with R₁₀ or R₁₃;
R₈ is H, —C₁₋₄ alkyl optionally substituted 1-3 times with R₁₀, or —C₃₋₆ cycloalkyl optionally substituted 1-3 times with R₁₀;
R₉ is H, halogen, —CN, —C₀₋₃ alkyl-C₃₋₆ cycloalkyl, or —C₁₋₆ alkyl optionally substituted 1-3 times with R₁₀;
R₁₀ is independently at each occurrence halogen, oxygen, hydroxy, —C₁₋₄ alkyl, or —O—C₁₋₄ alkyl;
R₁₁ and R₁₂ are each independently H, —C₁₋₄ alkyl, or —C₁₋₄ heteroalkyl, wherein R₁₁ and R₁₂ may combine to form a heterocycloalkyl; and
R₁₃ is independently at each occurrence —N(CH₃)₂ or

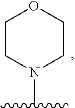 [SMILES: *N1CCOCC1]

or a pharmaceutically acceptable salt thereof; and
an effective amount of an Aurora A inhibitor, or a pharmaceutically acceptable salt thereof.
Embodiment 2. A compound of the formula:

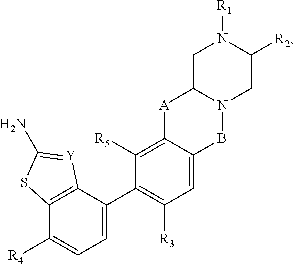

wherein:
A is —OCH₂—, —N(R₆)CH₂—, —OCH₂CH₂—, —N(R₆)CH₂CH₂—, —CH₂OCH₂—, or —CH₂N(R₆)CH₂—;
B is —CH₂— or —C(O)—;
Y is —C(CN)— or —N—;
R₁ is —CN, —C(O)C≡CR₈, or a group of the formula

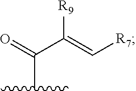

R₂ is H, methyl, or —CH₂CN;
R₃ and R₅ are each independently H, halogen, —C₀₋₃ alkyl-cyclopropyl, —C₁₋₆ alkyl optionally substituted 1-3 times with R₁₀, or —O—C₁₋₆ alkyl optionally substituted 1-3 times with R₁₀;
R₄ is H, halogen, or —C₁₋₆ alkyl optionally substituted 1-3 times with R₁₀;
R₆ is H or —C₁₋₆ alkyl optionally substituted 1-3 times with R₁₀;
R₇ is H, halogen, —NR₁₁R₁₂, —CH₂NR₁₁R₁₂, —C₁₋₆ alkyl optionally substituted 1-3 times with
R₁₀ or R₁₃, —C₀₋₃ alkyl cyclopropyl, or —O—C₁₋₆ alkyl optionally substituted 1-3 times with R₁₀ or R₁₃;
R₈ is H, —C₁₋₄ alkyl optionally substituted 1-3 times with R₁₀, or —C₃₋₆ cycloalkyl optionally substituted 1-3 times with R₁₀;
R₉ is H, halogen, —CN, —C₀₋₃ alkyl-C₃₋₆ cycloalkyl, or —C₁₋₆ alkyl optionally substituted 1-3 times with R₁₀;
R₁₀ is independently at each occurrence halogen, oxygen, hydroxy, —C₁₋₄ alkyl, or —O—C₁₋₄ alkyl;
R₁₁ and R₁₂ are each independently H, —C₁₋₄ alkyl, or —C₁₋₄ heteroalkyl, wherein R₁₁ and R₁₂ may combine to form a heterocycloalkyl; and
R₁₃ is independently at each occurrence —N(CH₃)₂ or

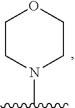 [SMILES: *N1CCOCC1]

or a pharmaceutically acceptable salt thereof, for use in simultaneous, separate, or sequential combination with an Aurora A inhibitor, or a pharmaceutically acceptable salt thereof, in therapy.
Embodiment 3. A compound of the formula:

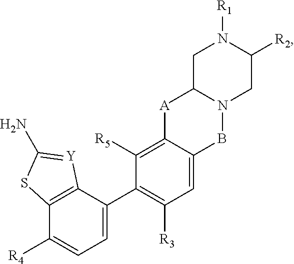

wherein:
A is —OCH₂—, —N(R₆)CH₂—, —OCH₂CH₂—, —N(R₆)CH₂CH₂—, —CH₂OCH₂—, or —CH₂N(R₆)CH₂—;
B is —CH₂— or —C(O)—;
Y is —C(CN)— or —N—;
R₁ is —CN, —C(O)C≡CR₈, or a group of the formula

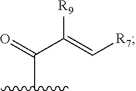

R₂ is H, methyl, or —CH₂CN;
R₃ and R₅ are each independently H, halogen, —C₀₋₃ alkyl-cyclopropyl, —C₁₋₆ alkyl optionally substituted 1-3 times with R₁₀, or —O—C₁₋₆ alkyl optionally substituted 1-3 times with R₁₀;
R₄ is H, halogen, or —C₁₋₆ alkyl optionally substituted 1-3 times with R₁₀;
R₆ is H or —C₁₋₆ alkyl optionally substituted 1-3 times with R₁₀;
R₇ is H, halogen, —NR₁₁R₁₂, —CH₂NR₁₁R₁₂, —C₁₋₆ alkyl optionally substituted 1-3 times with
R₁₀ or R₁₃, —C₀₋₃ alkyl cyclopropyl, or —O—C₁₋₆ alkyl optionally substituted 1-3 times with R₁₀ or R₁₃;
R₈ is H, —C₁₋₄ alkyl optionally substituted 1-3 times with R₁₀, or —C₃₋₆ cycloalkyl optionally substituted 1-3 times with R₁₀;
R₉ is H, halogen, —CN, —C₀₋₃ alkyl-C₃₋₆ cycloalkyl, or —C₁₋₆ alkyl optionally substituted 1-3 times with R₁₀;
R₁₀ is independently at each occurrence halogen, oxygen, hydroxy, —C₁₋₄ alkyl, or —O—C₁₋₄ alkyl;
R₁₁ and R₁₂ are each independently H, —C₁₋₄ alkyl, or —C₁₋₄ heteroalkyl, wherein R₁₁ and R₁₂ may combine to form a heterocycloalkyl; and
R₁₃ is independently at each occurrence —N(CH₃)₂ or,

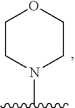 [SMILES: *N1CCOCC1]

or a pharmaceutically acceptable salt thereof, for use in simultaneous, separate, or sequential combination with an Aurora A inhibitor, or a pharmaceutically acceptable salt thereof, in the treatment of cancer.
Embodiment 4. Use of a compound of the formula:

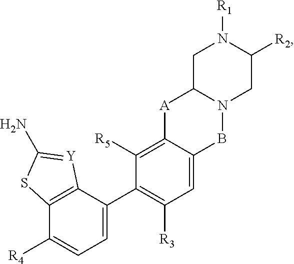

wherein:
A is —OCH₂—, —N(R₆)CH₂—, —OCH₂CH₂—, —N(R₆)CH₂CH₂—, —CH₂OCH₂—, or —CH₂N(R₆)CH₂—;
B is —CH₂— or —C(O)—;
Y is —C(CN)— or —N—;
R₁ is —CN, —C(O)C≡CR₈, or a group of the formula

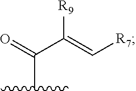

R₂ is H, methyl, or —CH₂CN;
R₃ and R₅ are each independently H, halogen, —C₀₋₃ alkyl-cyclopropyl, —C₁₋₆ alkyl optionally substituted 1-3 times with R₁₀, or —O—C₁₋₆ alkyl optionally substituted 1-3 times with R₁₀;
R₄ is H, halogen, or —C₁₋₆ alkyl optionally substituted 1-3 times with R₁₀;
R₆ is H or —C₁₋₆ alkyl optionally substituted 1-3 times with R₁₀;
R₇ is H, halogen, —NR₁₁R₁₂, —CH₂NR₁₁R₁₂, —C₁₋₆ alkyl optionally substituted 1-3 times with
R₁₀ or R₁₃, —C₀₋₃ alkyl cyclopropyl, or —O—C₁₋₆ alkyl optionally substituted 1-3 times with R₁₀ or R₁₃;
R₈ is H, —C₁₋₄ alkyl optionally substituted 1-3 times with R₁₀, or —C₃₋₆ cycloalkyl optionally substituted 1-3 times with R₁₀;
R₉ is H, halogen, —CN, —C₀₋₃ alkyl-C₃₋₆ cycloalkyl, or —C₁₋₆ alkyl optionally substituted 1-3 times with R₁₀;
R₁₀ is independently at each occurrence halogen, oxygen, hydroxy, —C₁₋₄ alkyl, or —O—C₁₋₄ alkyl;
R₁₁ and R₁₂ are each independently H, —C₁₋₄ alkyl, or —C₁₋₄ heteroalkyl, wherein R₁₁ and R₁₂ may combine to form a heterocycloalkyl; and
R₁₃ is independently at each occurrence —N(CH₃)₂ or,

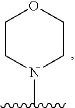 [SMILES: *N1CCOCC1]

or a pharmaceutically acceptable salt thereof, in simultaneous, separate, or sequential combination with an Aurora A inhibitor, or a pharmaceutically acceptable salt thereof, in the manufacture of a medicament for the treatment of cancer.
Embodiment 5. The method, compound, or use according to any one of embodiments 1-4, wherein A is —OCH₂CH₂—, or a pharmaceutically acceptable salt thereof.
Embodiment 6. The method, compound, or use according to any one of embodiments 1-5, wherein B is —C(O)—, or a pharmaceutically acceptable salt thereof.
Embodiment 7. The method, compound, or use according to any one of embodiments 1-6, wherein Y is —C(CN)—, or a pharmaceutically acceptable salt thereof.
Embodiment 8. The method, compound, or use according to any one of embodiments 1-6, wherein Y is —N—, or a pharmaceutically acceptable salt thereof.
Embodiment 9. The method, compound, or use according to any one of embodiments 1-8, wherein R₁ is a group of the formula

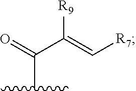

and wherein R₇ is H, F, Cl, methyl, ethoxy, ethyl, isopropyl, or cyclopropyl, or a pharmaceutically acceptable salt thereof.
Embodiment 10. The method, compound, or use according to any one of embodiments 1-9, wherein R₁ is a group of the formula

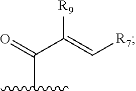

an wherein R₉ is H, F, Cl, —CHF₂, —CF₃, or —CH₂OH, or a pharmaceutically acceptable salt thereof.
Embodiment 11. The method, compound, or use according to any one of embodiments 1-8, wherein R₁ is —CN, or —C(O)C≡CR₈, or a pharmaceutically acceptable salt thereof.
Embodiment 12. The method, compound, or use according to any one of embodiments 1-11, wherein R₂ is H or methyl, or a pharmaceutically acceptable salt thereof.
Embodiment 13. The method, compound, or use according to any one of embodiments 1-12, wherein R₃ is H, F, Cl, methyl, methoxy, ethyl, isopropyl, or cyclopropyl, or a pharmaceutically acceptable salt thereof.
Embodiment 14. The method, compound, or use according to any one of embodiments 1-13, wherein R₄ is H, F, or Cl, or a pharmaceutically acceptable salt thereof.
Embodiment 15. The method, compound, or use according to any one of embodiments 1-14, wherein R₅ is H, —CHF₂, —CH₂F, —CH₂OH, or —CH₂OCH₃, or a pharmaceutically acceptable salt thereof.
Embodiment 16. The method, compound, or use according to any one of embodiments 1-15, wherein the compound is of the formula:

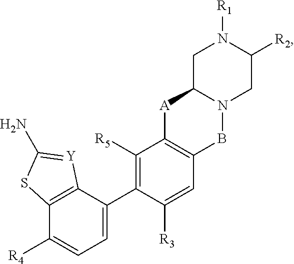

or a pharmaceutically acceptable salt thereof.
Embodiment 17. The method, compound, or use according to any one of embodiments 1-4, 6, 9, 10, 12-16, wherein the compound is of the formula:

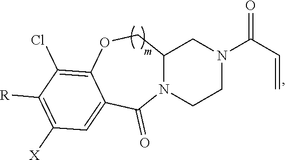

wherein R is

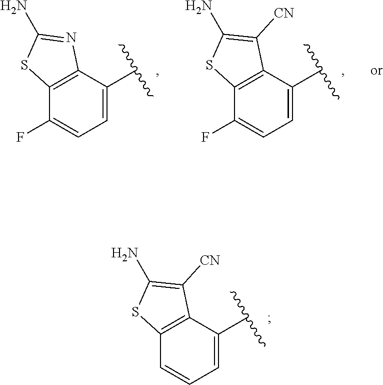 [SMILES: *c1ccc(F)c2sc(N)nc12 ; *c1ccc(F)c2sc(N)c(C#N)c12 ; *c1cccc2sc(N)c(C#N)c12]

X is Cl or F;
and m is 1 or 2,
or a pharmaceutically acceptable salt thereof.
Embodiment 18. The method, compound, or use according any one of embodiments 1-4, 6, 9, 10, 12-17, wherein the compound is of the formula:

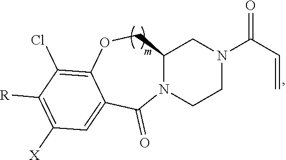

wherein R is

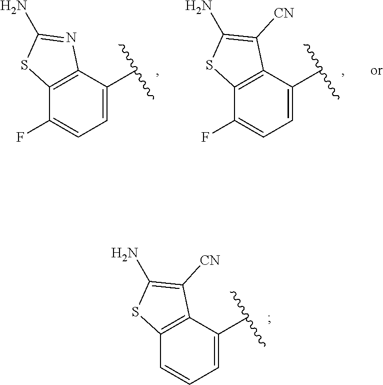 [SMILES: *c1ccc(F)c2sc(N)nc12 ; *c1ccc(F)c2sc(N)c(C#N)c12 ; *c1cccc2sc(N)c(C#N)c12]

X is Cl or F;
and m is 1 or 2,
or a pharmaceutically acceptable salt thereof.
Embodiment 19. The method, compound, or use according any one of embodiments 1-4, 6, 9, 10, 12-16, wherein the compound is of the formula:

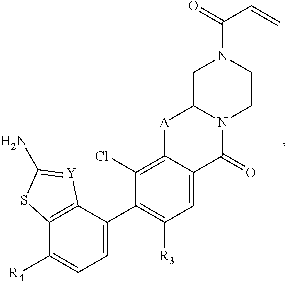

wherein:
A is —OCH₂— or —OCH₂CH₂—;
Y is C(CN) or N;
R₃ is Cl or F;
R₄ is H or F when Y is C(CN); and
R₄ is F when Y is N,
or a pharmaceutically acceptable salt thereof.
Embodiment 20. The method, compound, or use according any one of embodiments 1-4 or 6-19, wherein A is

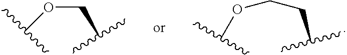

Embodiment 21. The method, compound, or use according any one of embodiments 1-4, 6, 9, 10, 12-20, wherein the compound is:

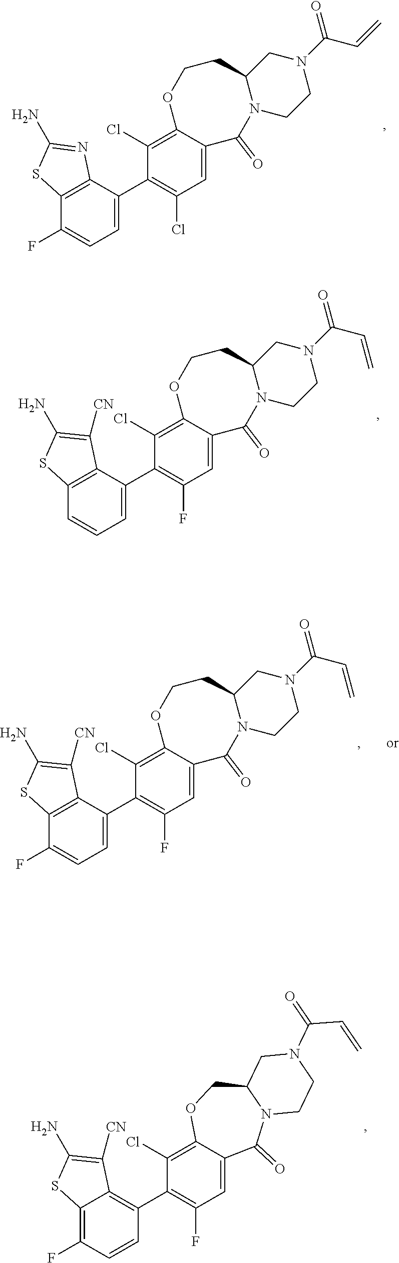 [SMILES: C=CC(=O)N1CCN2C(=O)c3cc(Cl)c(-c4ccc(F)c5sc(N)nc45)c(Cl)c3OCC[C@H]2C1 ; C=CC(=O)N1CCN2C(=O)c3cc(F)c(-c4cccc5sc(N)c(C#N)c45)c(Cl)c3OCC[C@H]2C1 ; C=CC(=O)N1CCN2C(=O)c3cc(F)c(-c4ccc(F)c5sc(N)c(C#N)c45)c(Cl)c3OCC[C@H]2C1 ; C=CC(=O)N1CCN2C(=O)c3cc(F)c(-c4ccc(F)c5sc(N)c(C#N)c45)c(Cl)c3OC[C@H]2C1]

or a pharmaceutically acceptable salt thereof.
Embodiment 22. The method, compound, or use according any one of embodiments 1-4, 6, 9, 10, 12-21, wherein the compound is:

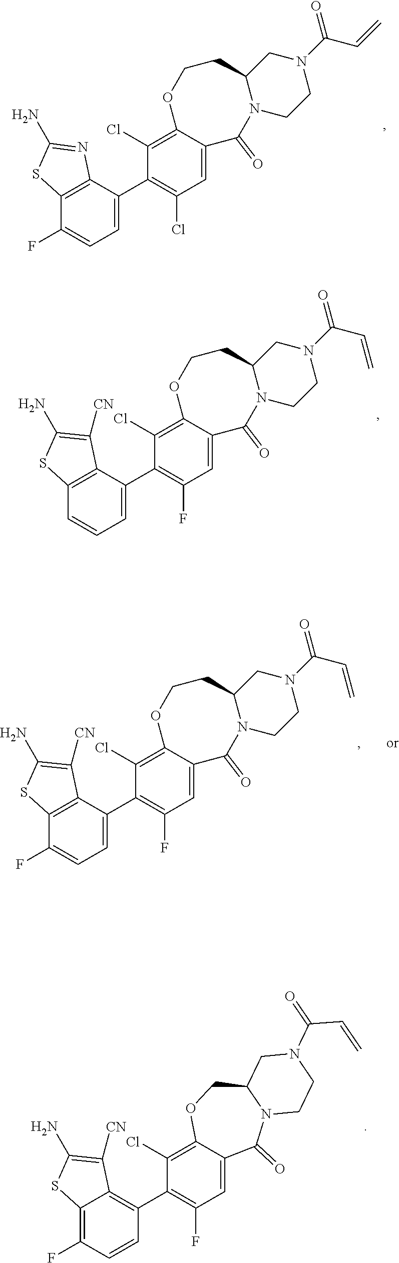 [SMILES: C=CC(=O)N1CCN2C(=O)c3cc(Cl)c(-c4ccc(F)c5sc(N)nc45)c(Cl)c3OCC[C@H]2C1 ; C=CC(=O)N1CCN2C(=O)c3cc(F)c(-c4cccc5sc(N)c(C#N)c45)c(Cl)c3OCC[C@H]2C1 ; C=CC(=O)N1CCN2C(=O)c3cc(F)c(-c4ccc(F)c5sc(N)c(C#N)c45)c(Cl)c3OCC[C@H]2C1 ; C=CC(=O)N1CCN2C(=O)c3cc(F)c(-c4ccc(F)c5sc(N)c(C#N)c45)c(Cl)c3OC[C@H]2C1]

Embodiment 23. The method, compound, or use according to any one of embodiments 1-4, 6, 9, 10, 12-22, wherein the compound is:

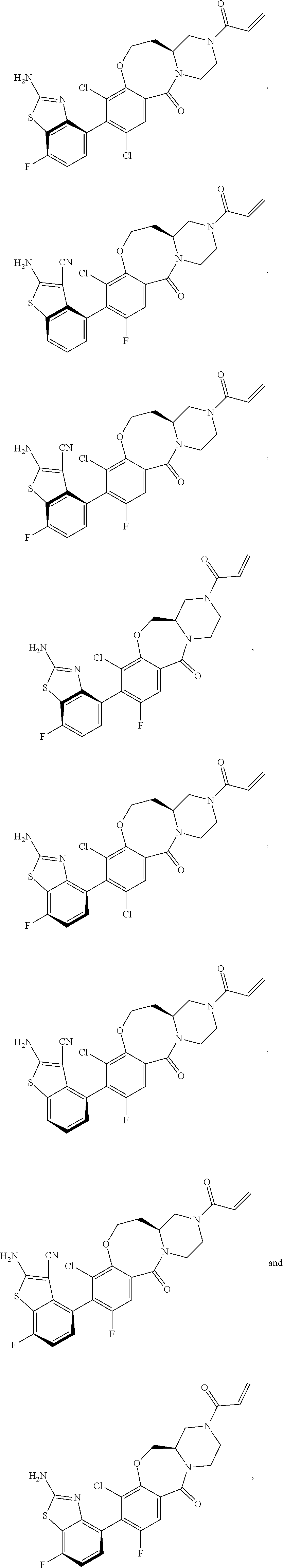

or a pharmaceutically acceptable salt thereof.
Embodiment 24. The method, compound, or use according to embodiment 23 selected from the group consisting of:

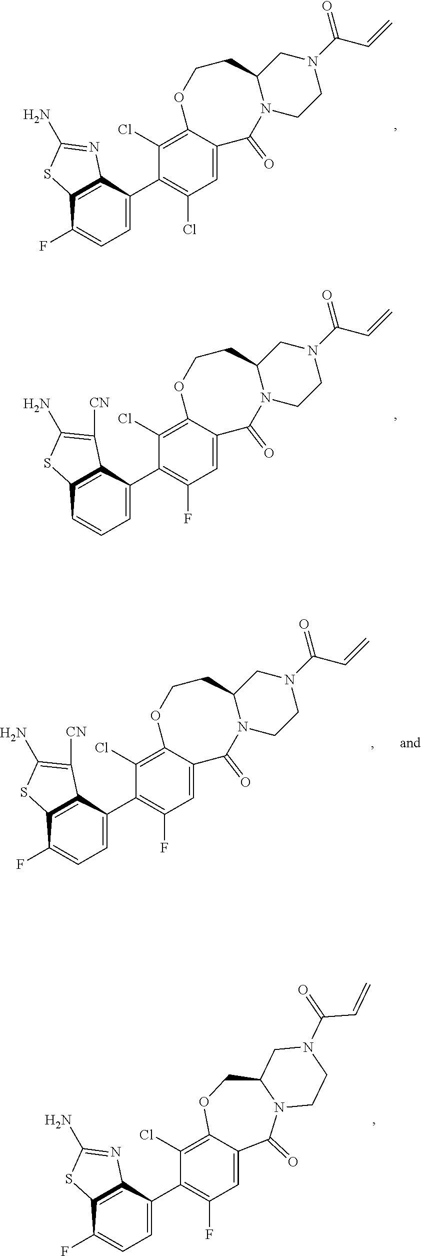

or a pharmaceutically acceptable salt thereof.
Embodiment 25. The method, compound, or use according to embodiments 23 or 24 selected from the group consisting of:

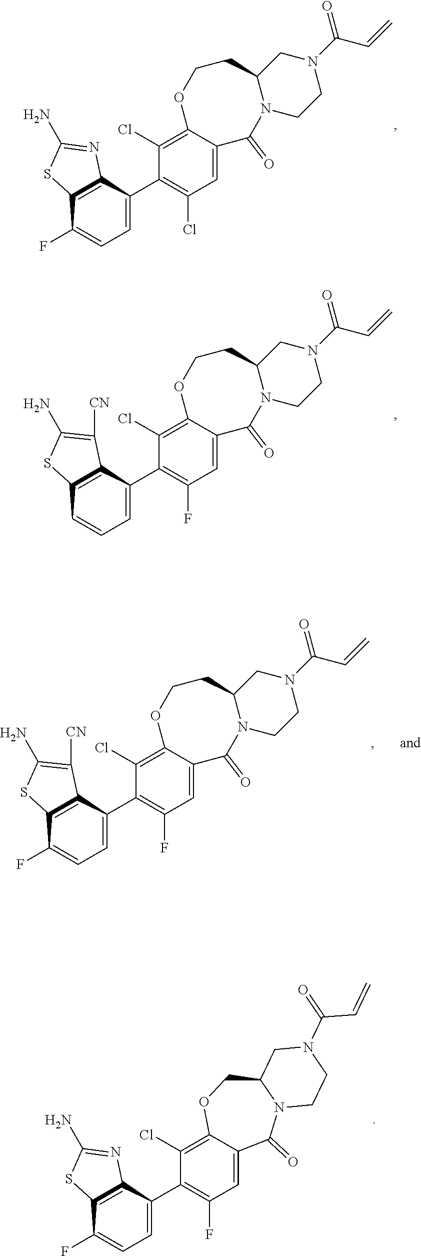

Embodiment 26. The method, compound, or use according to embodiment 23 selected from the group consisting of:

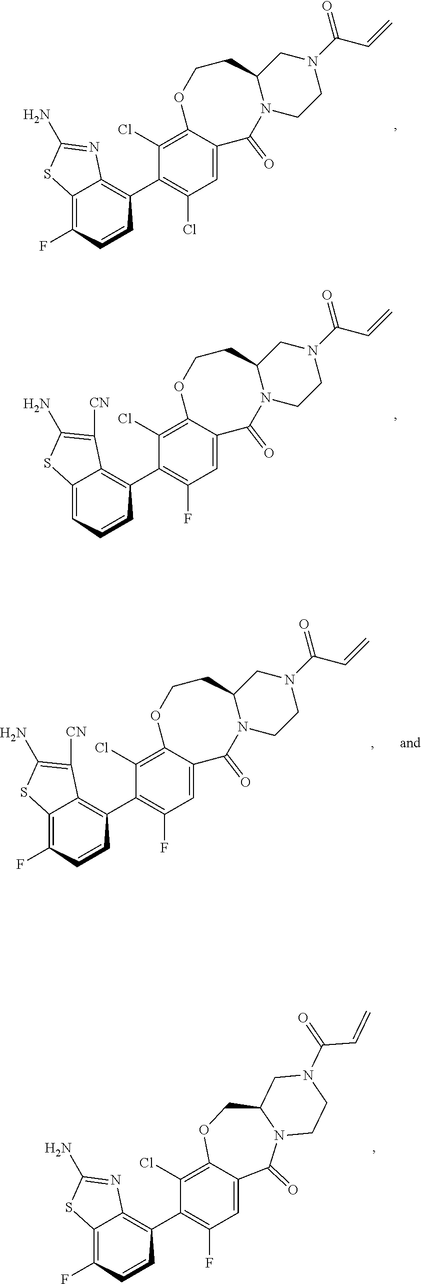

or a pharmaceutically acceptable salt thereof.
Embodiment 27. The method, compound, or use according to embodiments 23 or 26 selected from the group consisting of:

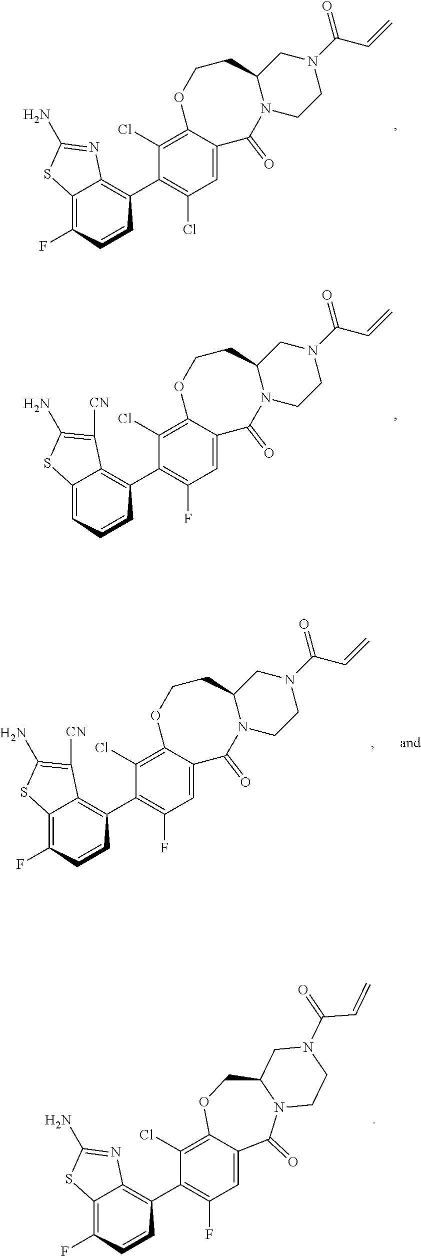

Embodiment 28. The method, compound, or use according to embodiment 23 selected from the group consisting of:

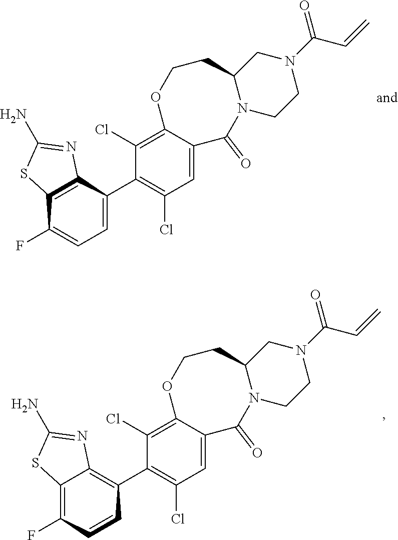

or a pharmaceutically acceptable salt thereof.
Embodiment 29. The method, compound, or use according to any one of embodiments 23, 24, 25, or 28 wherein the compound is

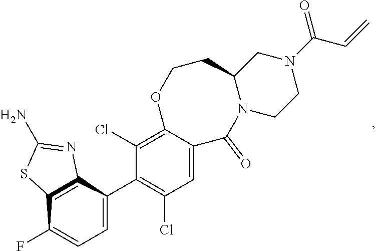

or a pharmaceutically acceptable salt thereof.
Embodiment 30. The method, compound, or use according to any one of embodiment 23, 26, 27, or 28 wherein the compound is

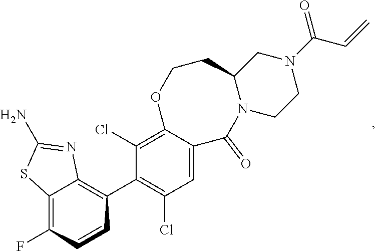

or a pharmaceutically acceptable salt thereof.
Embodiment 31. The method, compound, or use according to embodiment 23 selected from the group consisting of:

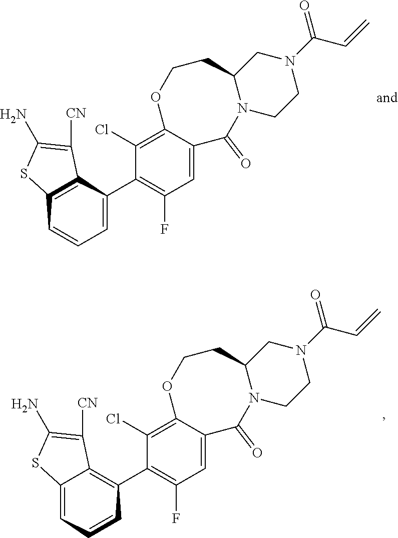

or a pharmaceutically acceptable salt thereof.
Embodiment 32. The method, compound, or use according to any one of embodiments 23, 24, 25 or 31 wherein the compound is

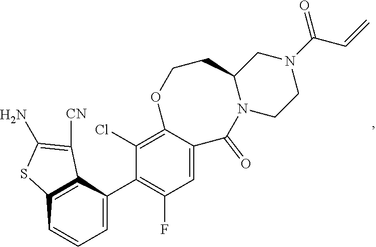

or a pharmaceutically acceptable salt thereof.
Embodiment 33. The method, compound, or use according to any one of embodiments 23, 26, 27, or 31 wherein the compound is

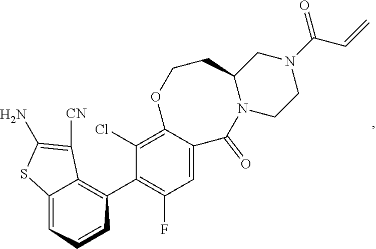

or a pharmaceutically acceptable salt thereof.
Embodiment 34. The method, compound, or use according to embodiment 23 selected from the group consisting of:

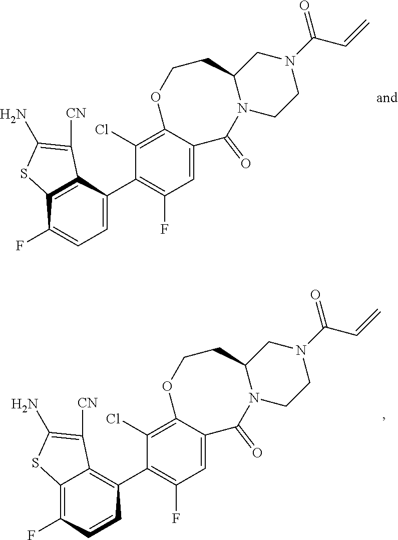

or a pharmaceutically acceptable salt thereof.
Embodiment 35. The method, compound, or use according to any one of embodiments 23, 24, 25, or 34 wherein the compound is

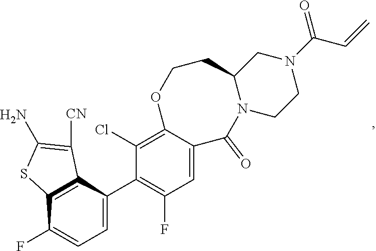

or a pharmaceutically acceptable salt thereof.
Embodiment 36. The method, compound, or use according to any one of embodiments 23, 26, 27, or 34 wherein the compound is

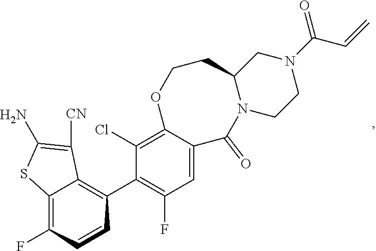

or a pharmaceutically acceptable salt thereof.
Embodiment 37. The method, compound, or use according to embodiment 23 selected from the group consisting of:

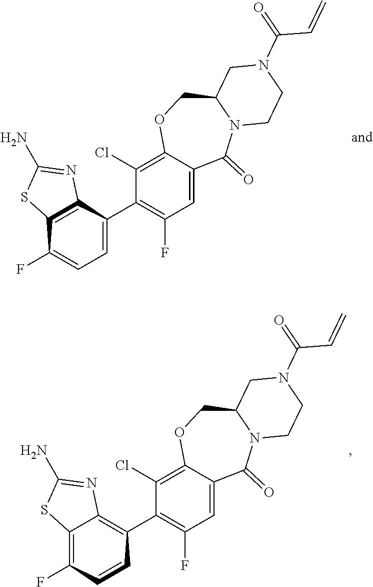

or a pharmaceutically acceptable salt thereof.
Embodiment 38. The method, compound, or use according to any one of embodiments 23, 24, 25, or 37 wherein the compound is

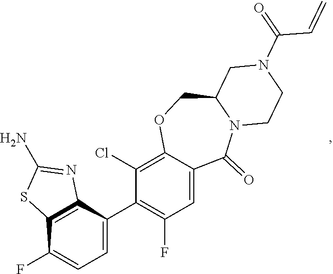

or a pharmaceutically acceptable salt thereof.
Embodiment 39. The method, compound, or use according to any one of embodiments 23, 26, 27, or 37 wherein the compound is

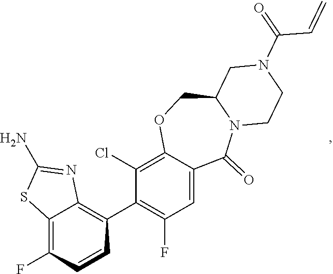

or a pharmaceutically acceptable salt thereof.
Embodiment 40. The method, compound, or use according to any one of embodiments 1-0, wherein the Aurora A inhibitor is selected from the group consisting of an Aurora A selective inhibitor, or a pharmaceutically acceptable salt thereof, alisertib, a pan Aurora inhibitor, or a pharmaceutically acceptable salt thereof, tozasertib, danusertib, an aminopyridine compound, or a pharmaceutically acceptable salt thereof, (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid, or a pharmaceutically acceptable salt thereof, (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:2-methylpropan-2-amine (1:1) salt, and (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:amine (1:1) salt.
Embodiment 41. The method, compound, or use according to any one of embodiments 1-40, wherein the Aurora A inhibitor is (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid, or a pharmaceutically acceptable salt thereof.
Embodiment 42. The method, compound, or use according to any one of embodiments 1-41, wherein the Aurora A inhibitor is selected from the group consisting of (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid, (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:2-methylpropan-2-amine (1:1) salt, and (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:amine (1:1) salt.
Embodiment 43. The method, compound, or use according to any one of embodiments 1-42, wherein the Aurora A inhibitor is (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:2-methylpropan-2-amine (1:1) salt.
Embodiment 44. A method, compound, or use according to any one of embodiments 1-43, wherein the cancer is selected from the group consisting of lung cancer, pancreatic cancer, cervical cancer, esophageal cancer, endometrial cancer, ovarian cancer, cholangiocarcinoma, and colorectal cancer.
Embodiment 45. The method, compound, or use according to any one of embodiments 1-44, wherein the cancer is non-small cell lung cancer, and wherein one or more cells express KRas G12C mutant protein with or without an Aurora A dysregulation or overexpression.
Embodiment 46. The method, compound, or use according to any one of embodiments 1-44, wherein the cancer is colorectal cancer, and wherein one or more cells with or without an Aurora A dysregulation or overexpression express KRas G12C mutant protein.
Embodiment 47. The method, compound, or use according to any one of embodiments 1-44, wherein the cancer is pancreatic cancer, and wherein one or more cells with or without an Aurora A dysregulation or overexpression express KRas G12C mutant protein.
Embodiment 48. The method, compound, or use according to any one of embodiments 1-44, wherein the patient has a cancer that has a KRAS G12C mutation.
Embodiment 49. The method, compound, or use according to any one of embodiments 1-48, wherein the patient has a cancer that was determined to have one or more cells expressing the KRas G12C mutant protein prior to administration of the compound, or a pharmaceutically acceptable salt thereof, or the Aurora A inhibitor, or a pharmaceutically acceptable salt thereof.
Embodiment 50. The method, compound, or use according to any one of embodiments 1-49 wherein the compound and the Aurora A inhibitor are administered in simultaneous or sequential combination to the patient in need thereof.
Embodiment 51. The method, compound, or use according to any one of embodiments 1-50, wherein the compound of the formula, or pharmaceutically acceptable salt thereof, and the Aurora A inhibitor, or pharmaceutically acceptable salt thereof, are administered in simultaneous combination to the patient in need thereof.
Embodiment 52. The method, compound, or use according to any one of embodiments 1-50, wherein the compound of the formula, or pharmaceutically acceptable salt thereof, and the Aurora A inhibitor, or pharmaceutically acceptable salt thereof, are administered in sequential combination to the patient in need thereof.
Embodiment 53. The method, compound, or use according to any one of embodiments 1-52, wherein the compound of the formula, or pharmaceutically acceptable salt thereof, is administered to the patient in need thereof before the Aurora A inhibitor, or pharmaceutically acceptable salt thereof, is administered to the patient in need thereof.
Embodiment 54. The method, compound, or use according to any one of embodiments 1-52, wherein the Aurora A inhibitor, or pharmaceutically acceptable salt thereof, is administered to the patient in need thereof before the compound of the formula, or pharmaceutically acceptable salt thereof, is administered to the patient in need thereof.

1. A method of treating a patient for cancer wherein one or more cells express KRas G12C mutant protein, comprising administering to a patient in need thereof, an effective amount of a compound of the formula: 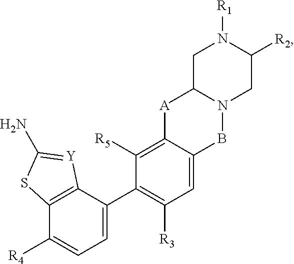
wherein:
A is —OCH₂—, —N(R₆)CH₂—, —OCH₂CH₂—, —N(R₆)CH₂CH₂—, —CH₂OCH₂—, or —CH₂N(R₆)CH₂—;
B is —CH₂— or —C(O)—;
Y is —C(CN)— or —N—;
R₁ is —CN, —C(O)C≡CR₈, or a group of the formula 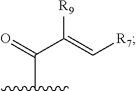
R₂ is H, methyl, or —CH₂CN;
R₃ and R₅ are each independently H, halogen, —C₀₋₃ alkyl-cyclopropyl, —C₁₋₆ alkyl optionally substituted 1-3 times with R₁₀, or —O—C₁₋₆ alkyl optionally substituted 1-3 times with R₁₀;
R₄ is H, halogen, or —C₁₋₆ alkyl optionally substituted 1-3 times with R₁₀;
R₆ is H or —C₁₋₆ alkyl optionally substituted 1-3 times with R₁₀;
R₇ is H, halogen, —NR₁₁R₁₂, —CH₂NR₁₁R₁₂, —C₁₋₆ alkyl optionally substituted 1-3 times with R₁₀ or R₁₃, —C₀₋₃ alkyl cyclopropyl, or —O—C₁₋₆ alkyl optionally substituted 1-3 times with R₁₀ or R₁₃;
R₈ is H, —C₁₋₄ alkyl optionally substituted 1-3 times with R₁₀, or —C₃₋₆ cycloalkyl optionally substituted 1-3 times with R₁₀;
R₉ is H, halogen, —CN, —C₀₋₃ alkyl-C₃₋₆ cycloalkyl, or —C₁₋₆ alkyl optionally substituted 1-3 times with R₁₀;
R₁₀ is independently at each occurrence halogen, oxygen, hydroxy, —C₁₋₄ alkyl, or —O—C₁₋₄ alkyl;
R₁₁ and R₁₂ are each independently H, —C₁₋₄ alkyl, or —C₁₋₄ heteroalkyl, wherein R₁₁ and R₁₂ may combine to form a heterocycloalkyl; and
R₁₃ is independently at each occurrence —N(CH₃)₂ or 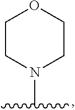 [SMILES: *N1CCOCC1]
or a pharmaceutically acceptable salt thereof; and
an effective amount of an Aurora A inhibitor, or a pharmaceutically acceptable salt thereof.

2. (canceled)

3. (canceled)

4. (canceled)

5. The method according to claim 1, wherein A is —OCH₂CH₂—, or a pharmaceutically acceptable salt thereof.

6. The method according to claim 1, wherein B is —C(O)—, or a pharmaceutically acceptable salt thereof.

7. The method according to claim 1, wherein Y is —C(CN)—, or a pharmaceutically acceptable salt thereof.

8. The method according to claim 1, wherein Y is —N—, or a pharmaceutically acceptable salt thereof.

9. The method according to claim 1, wherein R₁ is a group of the formula 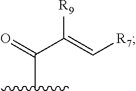
and wherein R₇ is H, F, Cl, methyl, ethoxy, ethyl, isopropyl, or cyclopropyl, or a pharmaceutically acceptable salt thereof.

10. The method according to claim 1, wherein R₁ is a group of the formula 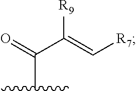
and wherein R₉ is H, F, Cl, —CHF₂, —CF₃, or —CH₂OH, or a pharmaceutically acceptable salt thereof.

11. The method according to claim 1, wherein R₁ is —CN, or —C(O)C≡CR₈, or a pharmaceutically acceptable salt thereof.

12. The method according to claim 1, wherein R₂ is H or methyl, or a pharmaceutically acceptable salt thereof.

13. The method according to claim 1, wherein R₃ is H, F, Cl, methyl, methoxy, ethyl, isopropyl, or cyclopropyl, or a pharmaceutically acceptable salt thereof.

14. The method according to claim 1, wherein R₄ is H, F, or Cl, or a pharmaceutically acceptable salt thereof.

15. The method according to claim 1, wherein R₅ is H, —CHF₂, —CH₂F, —CH₂OH, or —CH₂OCH₃, or a pharmaceutically acceptable salt thereof.

16. The method according to claim 1, wherein the compound is of the formula: 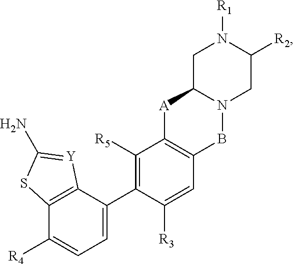
or a pharmaceutically acceptable salt thereof.

17. The method according to claim 1, wherein the compound is of the formula: 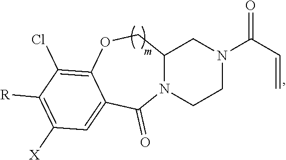
wherein R is or 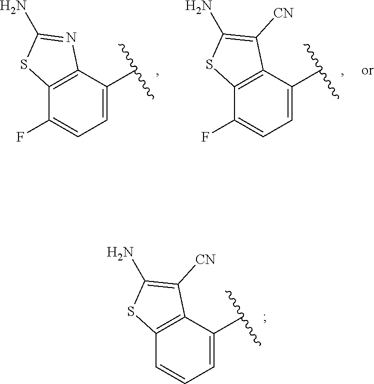 [SMILES: *c1ccc(F)c2sc(N)nc12 ; *c1ccc(F)c2sc(N)c(C#N)c12 ; *c1cccc2sc(N)c(C#N)c12]
X is Cl or F;
and m is 1 or 2,
or a pharmaceutically acceptable salt thereof.

18. The method according to claim 1, wherein the compound is of the formula: 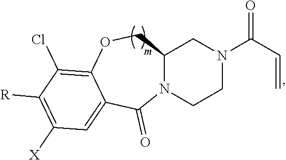
wherein R is 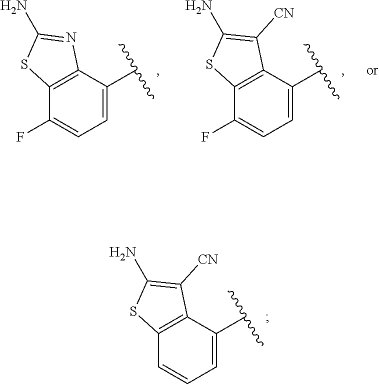 [SMILES: *c1ccc(F)c2sc(N)nc12 ; *c1ccc(F)c2sc(N)c(C#N)c12 ; *c1cccc2sc(N)c(C#N)c12]
X is Cl or F;
and m is 1 or 2,
or a pharmaceutically acceptable salt thereof.

19. The method according to claim 1, wherein the compound is of the formula: 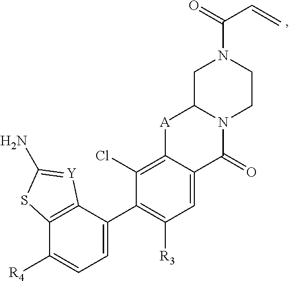
wherein:
A is —OCH₂— or —OCH₂CH₂—;
Y is C(CN) or N;
R₃ is Cl or F;
R₄ is H or F when Y is C(CN); and
R₄ is F when Y is N,
or a pharmaceutically acceptable salt thereof.

20. The method according to claim 1, wherein A is

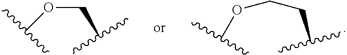

21. The method according to claim 1, wherein the compound is: 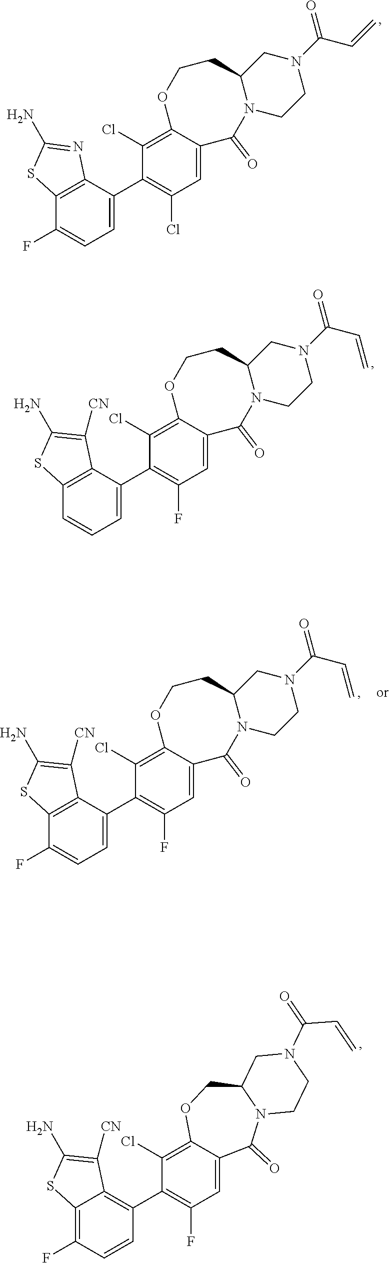 [SMILES: C=CC(=O)N1CCN2C(=O)c3cc(Cl)c(-c4ccc(F)c5sc(N)nc45)c(Cl)c3OCC[C@H]2C1 ; C=CC(=O)N1CCN2C(=O)c3cc(F)c(-c4cccc5sc(N)c(C#N)c45)c(Cl)c3OCC[C@H]2C1 ; C=CC(=O)N1CCN2C(=O)c3cc(F)c(-c4ccc(F)c5sc(N)c(C#N)c45)c(Cl)c3OCC[C@H]2C1 ; C=CC(=O)N1CCN2C(=O)c3cc(F)c(-c4ccc(F)c5sc(N)c(C#N)c45)c(Cl)c3OC[C@H]2C1]
or a pharmaceutically acceptable salt thereof.

22. The method according to claim 1, wherein the compound is:

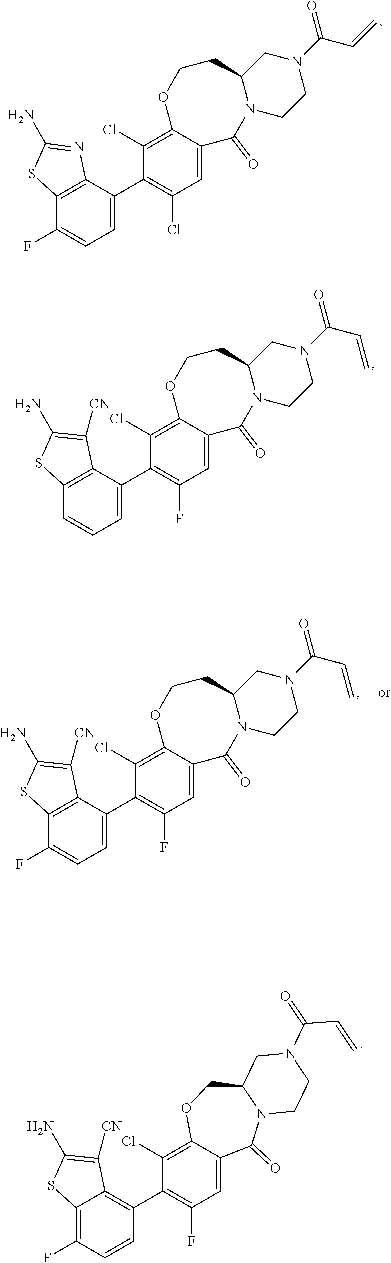 [SMILES: C=CC(=O)N1CCN2C(=O)c3cc(Cl)c(-c4ccc(F)c5sc(N)nc45)c(Cl)c3OCC[C@H]2C1 ; C=CC(=O)N1CCN2C(=O)c3cc(F)c(-c4cccc5sc(N)c(C#N)c45)c(Cl)c3OCC[C@H]2C1 ; C=CC(=O)N1CCN2C(=O)c3cc(F)c(-c4ccc(F)c5sc(N)c(C#N)c45)c(Cl)c3OCC[C@H]2C1 ; C=CC(=O)N1CCN2C(=O)c3cc(F)c(-c4ccc(F)c5sc(N)c(C#N)c45)c(Cl)c3OC[C@H]2C1]

23. The method according to claim 1, wherein the compound is selected from the group consisting of: 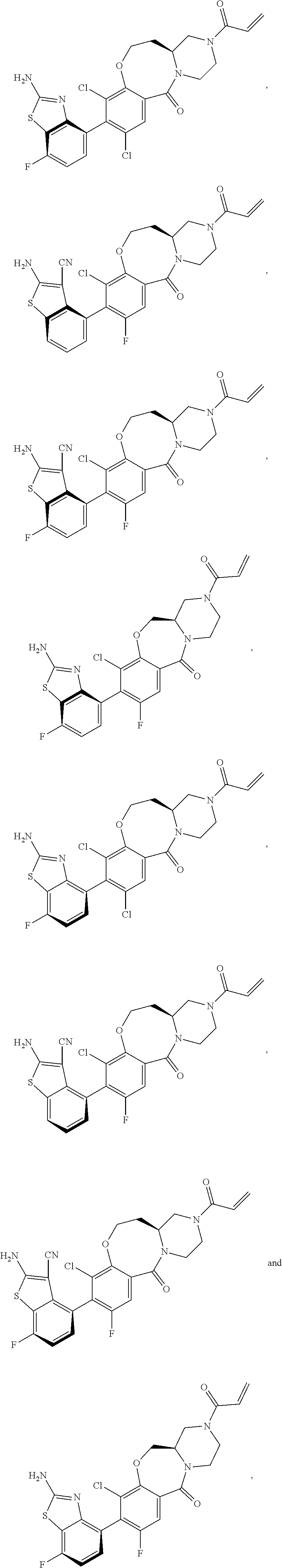
or a pharmaceutically acceptable salt thereof.

24. The method according to claim 23, wherein the compound is selected from the group consisting of: 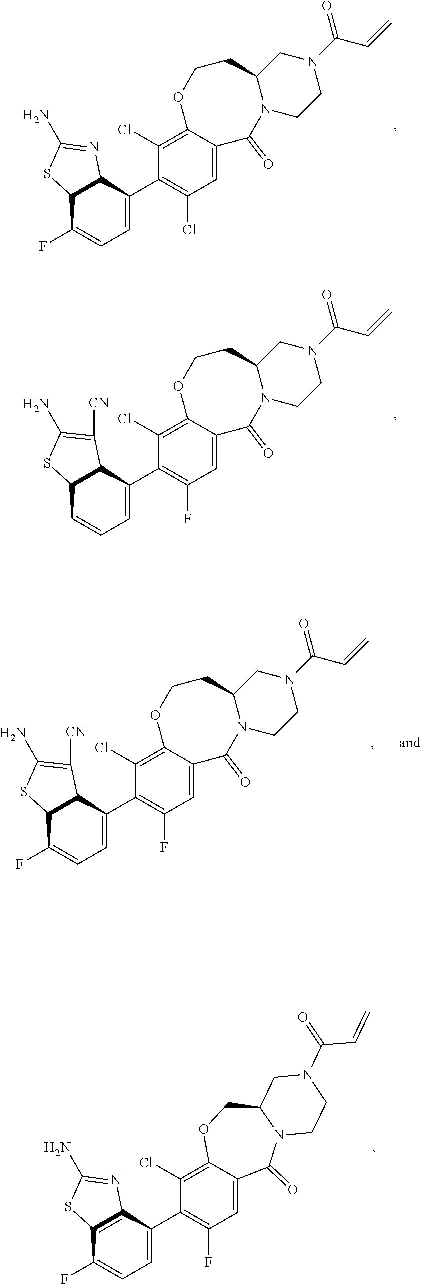
or a pharmaceutically acceptable salt thereof.

25. The method according to claim 24, wherein the compound is selected from the group consisting of:

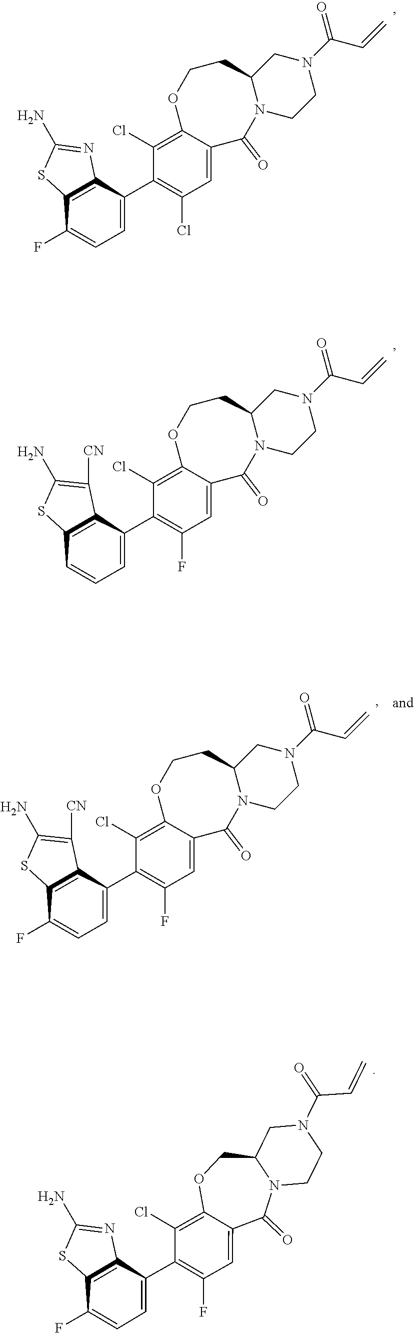

26. The method according to claim 23, wherein the compound is selected from the group consisting of: 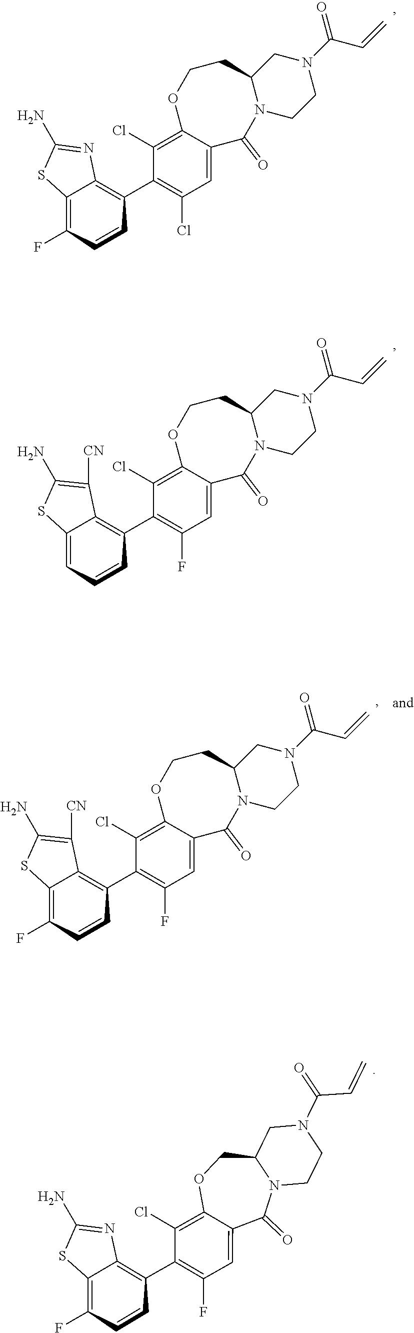
or a pharmaceutically acceptable salt thereof.

27. The method according to claim 26, wherein the compound is selected from the group consisting of:

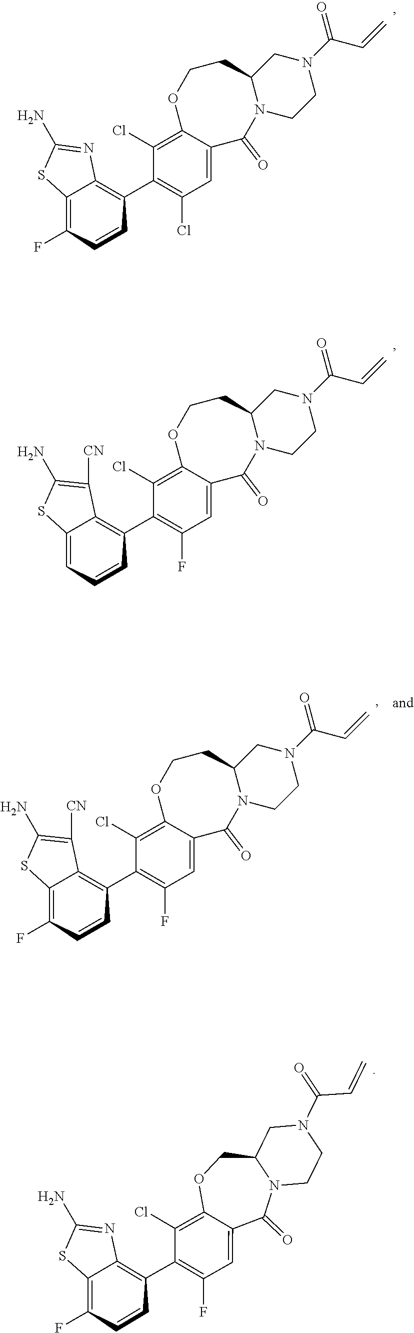

28. The method according to claim 23, wherein the compound is selected from the group consisting of: 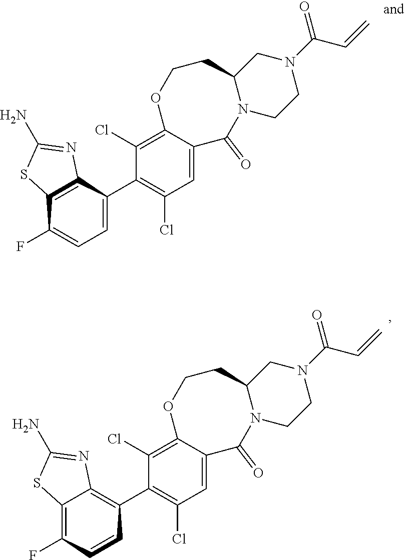
or a pharmaceutically acceptable salt thereof.

29. The method according to claim 23, wherein the compound is 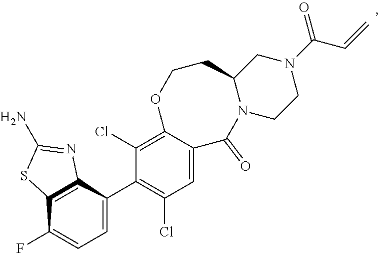
or a pharmaceutically acceptable salt thereof.

30. The method according to claim 23, wherein the compound is 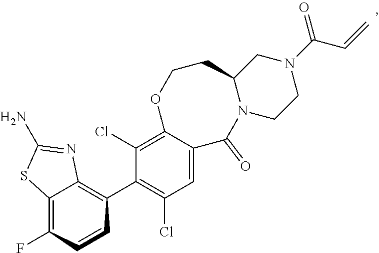
or a pharmaceutically acceptable salt thereof.

31. The method according to claim 23, wherein the compound is selected from the group consisting of 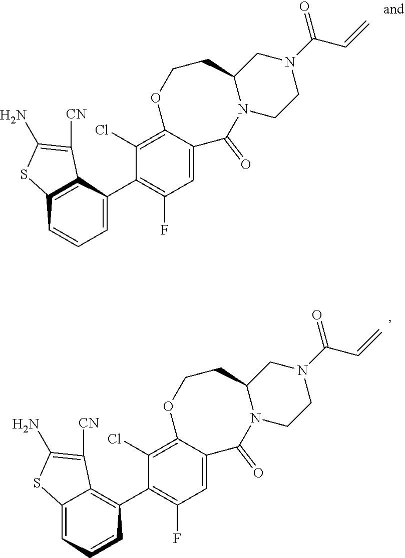
or a pharmaceutically acceptable salt thereof.

32. The method according to claim 23 wherein the compound is 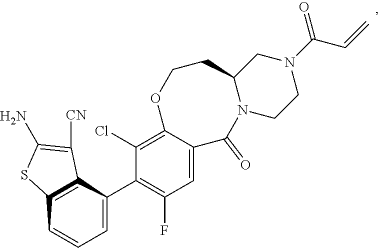
or a pharmaceutically acceptable salt thereof.

33. The method according to claim 23 wherein the compound is 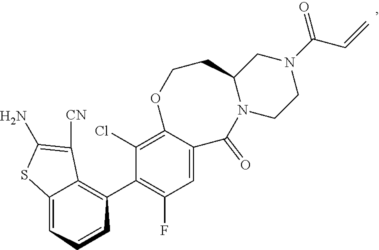
or a pharmaceutically acceptable salt thereof.

34. The method according to claim 23, wherein the compound is selected from the group consisting of: 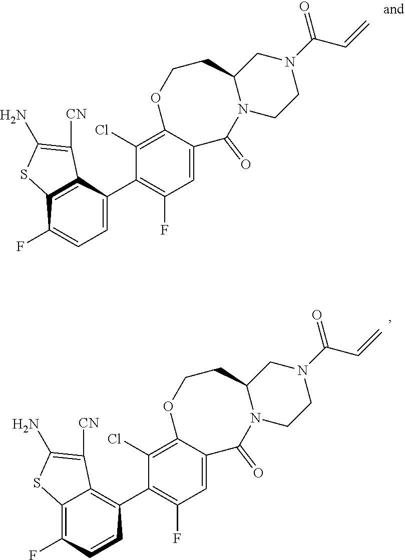
or a pharmaceutically acceptable salt thereof.

35. The method according to claim 23, wherein the compound is 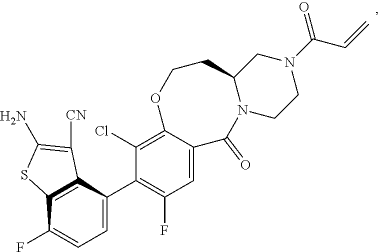
or a pharmaceutically acceptable salt thereof.

36. The method according to claim 23, wherein the compound is 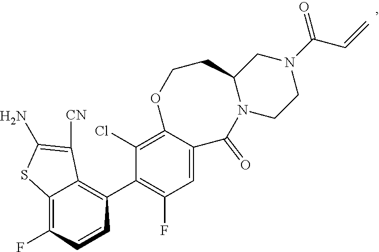
or a pharmaceutically acceptable salt thereof.

37. The method according to claim 23, wherein the compound is selected from the group consisting of: 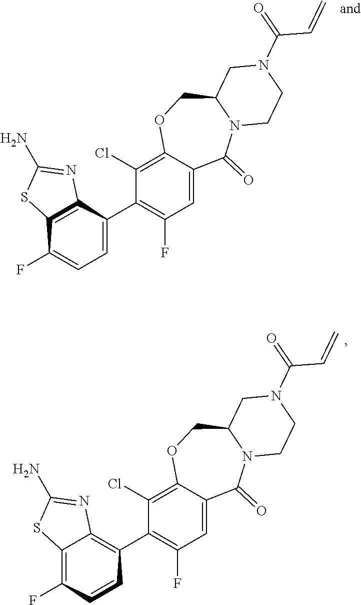
or a pharmaceutically acceptable salt thereof.

38. The method according to claim 23, wherein the compound is 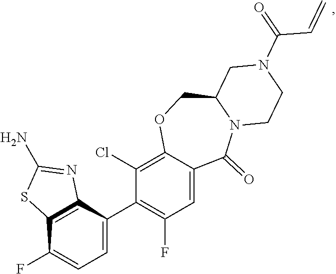
or a pharmaceutically acceptable salt thereof.

39. The method according to claim 23, wherein the compound is 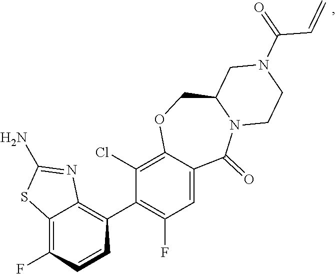
or a pharmaceutically acceptable salt thereof.

40. The method according to claim 1, wherein the Aurora A inhibitor is selected from the group consisting of an Aurora A selective inhibitor, or a pharmaceutically acceptable salt thereof, alisertib, a pan Aurora inhibitor, or a pharmaceutically acceptable salt thereof, tozasertib, danusertib, an aminopyridine compound, or a pharmaceutically acceptable salt thereof, (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid, or a pharmaceutically acceptable salt thereof, (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:2-methylpropan-2-amine (1:1) salt, and (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:amine (1:1) salt.

41. The method according to claim 1, wherein the Aurora A inhibitor is (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid, or a pharmaceutically acceptable salt thereof.

42. The method according to claim 1, wherein the Aurora A inhibitor is selected from the group consisting of (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid, (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:2-methylpropan-2-amine (1:1) salt, and (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:amine (1:1) salt.

43. The method according to claim 1, wherein the Aurora A inhibitor is (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:2-methylpropan-2-amine (1:1) salt.

44. A method according to claim 1, wherein the cancer is selected from the group consisting of lung cancer, pancreatic cancer, cervical cancer, esophageal cancer, endometrial cancer, ovarian cancer, cholangiocarcinoma, and colorectal cancer.

45. The method according to claim 1, wherein the cancer is non-small cell lung cancer, and wherein one or more cells express KRas G12C mutant protein with or without an Aurora A dysregulation or overexpression.

46. The method according to claim 1, wherein the cancer is colorectal cancer, and wherein one or more cells with or without an Aurora A dysregulation or overexpression express KRas G12C mutant protein.

47. The method according to claim 1, wherein the cancer is pancreatic cancer, and wherein one or more cells with or without an Aurora A dysregulation or overexpression express KRas G12C mutant protein.

48. The method according to claim 1, wherein the patient has a cancer that has a KRAS GT2C mutation.

49. The method according to claim 1, wherein the patient has a cancer that was determined to have one or more cells expressing the KRas G12C mutant protein prior to administration of the compound, or a pharmaceutically acceptable salt thereof, or the Aurora A inhibitor, or a pharmaceutically acceptable salt thereof.

50. The method according to claim 1, wherein the compound and the Aurora A inhibitor are administered in simultaneous or sequential combination to the patient in need thereof.

51. The method according to claim 1, wherein the compound of the formula, or pharmaceutically acceptable salt thereof, and the Aurora A inhibitor, or pharmaceutically acceptable salt thereof, are administered in simultaneous combination to the patient in need thereof.

52. The method according to claim 1, wherein the compound of the formula, or pharmaceutically acceptable salt thereof, and the Aurora A inhibitor, or pharmaceutically acceptable salt thereof, are administered in sequential combination to the patient in need thereof.

53. The method according to claim 1, wherein the compound of the formula, or pharmaceutically acceptable salt thereof, is administered to the patient in need thereof before the Aurora A inhibitor, or pharmaceutically acceptable salt thereof, is administered to the patient in need thereof.

54. The method according to claim 1, wherein the Aurora A inhibitor, or pharmaceutically acceptable salt thereof, is administered to the patient in need thereof before the compound of the formula, or pharmaceutically acceptable salt thereof, is administered to the patient in need thereof.
